(19) United States
(12) Patent Application Publication
(10) Pub. No.: US 2026/0345048 A1
(43) Pub. Date: Nov. 5, 2026

(54) APPARATUS, SYSTEMS, AND METHODS FOR HIGH-BANDWIDTH NEURAL INTERFACES

(71) Applicant: PRECISION NEUROSCIENCE CORPORATION, New York, NY (US)

(72) Inventors: Mark HETTICK, Fremont, CA (US); Demetrios Philip Papageorgiou, Weston, MA (US); Adam Poole, Brooklyn, NY (US); Benjamin Issac Rapoport, New York, NY (US)

(73) Assignee: PRECISION NEUROSCIENCE CORPORATION, New York, NY (US)

(21) Appl. No.: 19/742,483

(22) Filed: Jul. 15, 2026

Related U.S. Application Data
(63) Continuation of application No. 17/966,359, filed on Oct. 14, 2022.
(60) Provisional application No. 63/255,724, filed on Oct. 14, 2021.

(51) Int. Cl. A61B 5/293; A61B 5/00

(52) U.S. Cl. CPC A61B 5/293; A61B 5/4836; A61B 5/6862; A61B 2560/066; A61B 2562/0209; A61B 2562/028; A61B 2562/164; A61B 2562/222; A61B 2562/227

(57) ABSTRACT

Disclosed is a stent-mesh and microelectrode assembly that is deployable using a catheter or cannula to form a neural interface for recording and/or stimulation of neural tissue. In some embodiments, the assembly may include a thin-film microelectrode array attached to a spring-like stent-mesh component. The thin-film microelectrode array may include an electrode body having two lateral wing-like appendages located distal to a thin-film flexible cable that terminates at the proximal end in a thin-film connector region. The stent-mesh may be attached to the thin-film microelectrode array and configured to be advanced to a target area in a collapsed state and then expanded after reaching the target area to transition the thin-film microelectrode array to a deployed configuration. Accordingly, the assembly may deliver the thin-film microelectrode array to a target area in a minimally invasive manner.

### CROSS-REFERENCE TO RELATED APPLICATIONS

This application is a continuation of U.S. patent application Ser. No. 17/966,359, filed Oct. 14, 2022, which claims the benefit of priority to U.S. Provisional Application No. 63/255,724 entitled “Apparatus, Systems, and Methods for High-Bandwidth Neural Interfaces,” filed Oct. 14, 2021, the entireties of which are incorporated herein by reference.

### TECHNICAL FIELD

The present disclosure is directed towards high-bandwidth neural interfaces. More particularly, the present disclose relates to stent-mesh devices that may be used to deploy high-bandwidth neural interfaces. The disclosed techniques may be applied to, for example, deployment of high-bandwidth neural interfaces in a compressed configuration to a target site in a minimally invasive manner and transitioning the neural interfaces to an expanded configuration at the target site.

### BACKGROUND

Neural recording and stimulation techniques involve design trade-offs among (1) spatial resolution, (2) temporal resolution, (3) degree of invasiveness and collateral damage to normal brain tissue, and (4) optimization for electrical recording and/or electrical stimulation. Accordingly, there remains a need for high-bandwidth neural interfaces for the brain that allow for improved spatial resolution, temporal resolution, reduced collateral damage to the normal brain tissue and optimization for electrical recording and/or electrical stimulation.

Conventional techniques for recording and/or stimulating the nervous system face many challenges. For example, imaging techniques such as magnetic resonance imaging (MRI) and computed tomography (CT) provide non-invasive methods for examining brain tissue. However, these non-invasive imaging techniques are unable to detect all functional lesions, do not provide a method for imaging electrical activity in the nervous system, lack temporal resolution, and are unable to provide a mechanism for therapeutic electrophysiologic intervention.

Electromagnetic recording techniques such as electroencephalography (EEG) and magnetoencephalography (MEG) are non-invasive and provide temporal resolution of electrical activity in the brain. However, the resolution of such techniques is limited, due to both the physical distance separating the electrodes from the brain, and by the dielectric properties of the scalp and skull. Accordingly, there is a need for improved recording of neural activity with improved spatial resolution.

Additional techniques such as electrocorticography (ECoG) or intracranial EEG include forms of electroencephalography that provide improved spatial resolution by placing electrodes directly onto the cortical surface of the brain. However, the improved spatial resolution is conventionally achieved only by way of a highly invasive surgical procedure, a craniotomy, which requires the temporary surgical removal of a significant portion of the skull.

Another technique for recording and/or stimulating involves the use of depth electrodes which are capable of recording electrical activity in the nervous system with high spatial and temporal resolution. However, conventional depth electrodes are limited in that they are only able to record from a small volume or tissue, or a small population of neurons. Additionally, the placement of depth electrodes is highly invasive, and results in damage or destruction of normal brain tissue including neurons. Accordingly, the number of depth electrodes that can be safely placed is limited, as is the ability to adjust the spatial placement of the electrodes once they are placed, but for the minor adjustments to the depth of the electrode at the time of placement.

Another technique for recording and/or stimulating the nervous system includes the use of deep brain stimulation (DBS) electrodes, which may be configured to stimulate brain regions with millimetric and sub-millimetric precision. Although the DBS electrodes may be implanted leveraging minimally invasive surgical techniques, the electrode penetrates the brain which results in damage to the brain and carries risks including hemorrhage, stroke, and seizures. DBS electrodes can be used for stimulation as a way of treating conditions such as Parkinson's disease and essential tremor, and potentially some forms of epilepsy. However, the number of DBS electrodes that can safely be placed is limited, as is the ability to adjust the spatial placement of the electrodes, once they are placed. In practice, DBS techniques have an excellent safety profile demonstrated over two decades of standard clinical use, but as these electrodes are macroscopic, only a small number (typically one or two) are placed in any single patient.

Accordingly, conventional systems have limited the applications of neural recording and stimulation due to the invasiveness of delivery procedures for a small number of electrodes. As such, it would be advantageous to have delivery devices and systems for minimally invasively delivering high-bandwidth neural interfaces to a target site of a subject for neural recording and stimulation.

### SUMMARY

Disclosed herein are high-bandwidth neural interfaces for the brain that allow for improved spatial resolution, temporal resolution, reduced collateral damage to the normal brain tissue and optimization for electrical recording and/or electrical stimulation. Embodiments may include biocompatible, implantable devices that can be implanted into the brain to form a brain-computer interface. The neural interfaces may be used for recording and/or stimulation, including for example, for applying light, current, voltage, or drugs.

In some embodiments, the electrode arrays may be rollable or collapsible so that they are capable of being deployed through a narrow catheter or cannula and through one or more small burr holes. In some embodiments, the narrow catheter or canula has a diameter of less than or equal to 3 millimeters. In some embodiments, the burr holes may have a diameter of less than or equal to 3 millimeters. In some embodiments, the electrode arrays may be rollable or collapsible while being advanced to a target area. After reaching the target area, the electrode arrays may be expanded from their rolled or collapsed state.

Embodiments of the present disclosure may include a stent-mesh that is attached to a thin-film microelectrode array capable of recording from, and/or stimulating neural tissue. In some embodiments, the assembly may include a thin-film microelectrode array which is attached to a spring-like mesh. The thin-film microelectrode array may include an electrode body having two lateral wing-like appendages located distal to a thin-film flexible cable that terminates at the proximal end in a thin-film connector region. The stent-mesh may be attached to the thin-film microelectrode array configured to be advanced to a target area in a collapsed state and then expanded after reaching the target area.

In some embodiments, a neural interface device may include a thin-film microelectrode array configured to at least one of record from or stimulate a target area and a stent-mesh configured to attach to the thin-film microelectrode array, where the thin-film microelectrode array and stent-mesh form an assembly configured to have an expanded state and a rolled-up state. Optionally, the thin-film microelectrode array may include a connector, a thin-film flexible cable in electrical communication with the connector, and two lateral wings distal to the thin-film flexible cable. Optionally, the two lateral wings are flexible. The stent-mesh may include an eyelet and so may the thin-film microelectrode array. The neural interface may include one or more sutures configured to engage with the eyelet of the stent-mesh and the eyelet of the thin-film microelectrode array. In some embodiments, the connector may include a hermetically sealed feedthrough. Optionally, the thin-film microelectrode array may include active electronics. In some embodiments, the stent-mesh may be composed of a shape memory alloy, for example, nitinol. In some embodiments, at least one of the thin-film microelectrode array and the stent-mesh assembly may possess retrieval features configured to allow retrieval of the thin-film microelectrode array and the stent-mesh assembly by re-rolling into a delivery device cannula upon retraction. In some embodiments, the retrieval features may include a taper positioned at the proximal end of the electrode body.

In some embodiments, a neural interface may include a self-expanding thin-film microelectrode array configured to at least one of record from or stimulate a target area, the self-expanding thin-film microelectrode array comprising at least one retrieval feature configured to allow retrieval of the self-expanding thin-film microelectrode array by re-rolling into a delivery device upon retraction, and the self-expanding thin-film microelectrode array is configured to have an expanded and rolled-up state. Optionally, the retrieval feature includes a taper.

In some embodiments, a connector for neural interfaces include a biocompatible housing configured to receive conductive traces embedded in a flexible cable extending from a microelectrode array, a hermetically sealed feedthrough, an analog-to-digital converter, printed circuit board (PCB), and a multiplexer.

In some embodiments, a method for fabricating a microelectrode array include the steps of depositing a first polyimide layer onto a silicon wafer, generating at least one first insulating adhesion and barrier layer using chemical surface transformation or deposited coatings, depositing and patterning electrical traces onto the first polyimide and first insulation and barrier layers, generating a second insulating adhesion and barrier layer using chemical surface transformation or deposited coatings, patterning the second insulating adhesion and barrier layer, depositing a second polyimide layer onto the insulating layers and electrical traces, patterning the second polyimide layer, and depositing a pad metal layer onto the second polyimide layer.

In some embodiments, a method for fabricating a microelectrode array with embedded active electronics including depositing a first polyimide layer onto a silicon wafer, generating a first insulating adhesion and barrier layer using chemical transformation or deposited coatings, depositing and patterning electrical traces onto at least one of the first polyimide layer and the first insulation adhesion and barrier layer, generating a second insulating adhesion and barrier layer using chemical transformation or deposited coatings, patterning electrical traces onto the second insulating adhesion and barrier layer, generating circuits by depositing thin-film semiconductor layers onto the patterned electrical traces, generating a third insulating adhesion and barrier layer using chemical transformation or deposited coatings, patterning electrical traces onto the third insulating adhesion and barrier layer, depositing and patterning a second set of electrical traces onto the third insulating adhesion and barrier layer, creating a fourth insulating adhesion and barrier layer using chemical transformation or deposited coatings, patterning electrical traces onto the fourth insulating adhesion and barrier layer, depositing a second polyimide layer onto the fourth insulating adhesion and barrier layer and patterned electrical traces, patterning the second polyimide layer, and depositing a pad metal layer onto the second polyimide layer, wherein the generated circuits can be positioned along a substantially planar portion of the microelectrode array.

In some embodiments, a stent assembly may include a microelectrode array, and an expansible stent composed of nitinol and configured to attach to and provide mechanical support to the microelectrode array, where the expansible stent comprises a retracted state and a deployed state, where in the deployed state, the expansible stent forms a planar surface and wherein both the expansible stent and the microelectrode array are configured to conform to a cortical surface. In some embodiments, an expansible stent is configured to attach to the microelectrode array via a plurality of stitches.

In some embodiments, a method may include the steps of attaching a thin-film microelectrode array to a stent to form a stent-microelectrode assembly, rolling wings of the thin-film microelectrode array towards a central region of the thin-film microelectrode array, loading the stent-microelectrode assembly into a delivery catheter, advancing the delivery catheter to a target region, delivering the stent-microelectrode assembly at the target region, expanding the stent-microelectrode assembly, positioning the stent-microelectrode assembly adjacent to the target region, detaching the stent from the thin-film microelectrode array, and removing the stent from the target region via the delivery catheter.

### BRIEF DESCRIPTION OF THE DRAWINGS

The accompanying drawings, which are incorporated in and form a part of the specification, illustrate the embodiments of the invention and together with the written description serve to explain the principles, characteristics, and features of the invention. Various aspects of at least one example are discussed below with reference to the accompanying drawings, which are not intended to be drawn to scale. In the drawings:

FIG. 1A depicts a schematic diagram of a neural interface system in accordance with an embodiment.

FIG. 1B depicts an illustrative neural interface device including a thin-film microelectrode array in accordance with an embodiment.

FIG. 1C depicts an exemplary electrode array with wing features in accordance with an embodiment.

FIG. 1D depicts a schematic view of an exemplary neural interface system in accordance with an embodiment.

FIGS. 2A-2D depict several exemplary stent-mesh assemblies in accordance with various embodiments.

FIG. 2E depicts a bending test and design method for a stent or mesh for use with a microelectrode array in accordance with an embodiment.

FIG. 3A depicts an exemplary method for folding an electrode array and stent-mesh assembly in accordance with an embodiment.

FIG. 3B depicts an exemplary method for folding an electrode array and stent-mesh assembly in accordance with an embodiment.

FIG. 3C depicts deployment or unfurlment of an electrode array in accordance with an embodiment.

FIG. 3D depicts deployment or unfurlment of an electrode array and stent-mesh assembly in accordance with an embodiment.

FIG. 4A depicts a flow diagram of an exemplary method for fabricating a microelectrode array in accordance with an embodiment.

FIG. 4B depicts an exemplary layout for a wafer for microelectrode arrays in accordance an embodiment.

FIG. 4C depicts an exemplary microelectrode array in accordance with an embodiment.

FIG. 4D depicts an exemplary microelectrode array in accordance with an embodiment.

FIGS. 5A-5E depict a process for manufacturing the microelectrode array in accordance with an embodiment.

FIGS. 6A-6C depict several exemplary wafer designs used in connection with manufacturing a microelectrode array in accordance with various embodiments.

FIG. 7A depicts a schematic view of a thin-film based feedthrough of a microelectrode array in accordance with an embodiment.

FIG. 7B depicts a cross-sectional view of an encapsulated connector region within a catheter in accordance with an embodiment.

FIGS. 8A-8D depict several examples of a printed circuit board (PCB) and connector for a flexible surface microelectrode array in accordance with various embodiments.

FIGS. 9A-9C depict several views of a surface array layout for a microelectrode array in accordance with an embodiment.

FIGS. 10A-10D depict several exemplary surface array schematic layouts with multiplexing elements in accordance with various embodiments.

FIG. 11 illustrates a block diagram of an exemplary data processing system in which embodiments are implemented.

### DETAILED DESCRIPTION

As discussed herein, it may be advantageous to have delivery devices and systems for minimally invasively delivering high-bandwidth neural interfaces to a target site. High-bandwidth neural interfaces may include a high-channel count conformal electrode array. In some embodiments, the high-channel count conformal electrode array may be configured for the cortical surface. Alternatively, or additionally, in some embodiments the high-channel count conformal electrode array may be configured for intraventricular and endovascular areas.

Such minimally invasive neural interfaces may be useful for research and treatment of previously intractable medical conditions such as refractory epilepsy, blindness, paralysis, cognitive impairment and impulse-control disorders. This may include neural interfaces used from neuro-electrophysiology, mapping, recording, and stimulation of the brain and nervous systems. Additionally, neural interfaces may be used in the diagnosis, research, and treatment of conditions such as epilepsy and seizure disorders, paralysis associated with stroke or spinal cord injury, visual impairment, memory and cognitive impairment and human-to-computer interfaces.

It should be understood that neural interfaces may be used for the diagnosis, research and/or treatment of various conditions in which the electrical activity of the nervous system is dysregulated (e.g., epilepsy), electrical pathways of the nervous system are disrupted (e.g., stroke or traumatic injury), and/or electrical pathways cease to function (e.g., Parkinson's disease).

### Minimally Invasive Neural Interface Systems

The minimally invasive, high-bandwidth neural interfaces utilized with the systems herein are first described in greater detail. These neural interface systems may allow for improved spatial resolution, temporal resolution, reduced collateral damage to the normal brain tissue, and optimization for electrical recording and/or electrical stimulation. Embodiments may include biocompatible, implantable devices that may be implanted into the brain to form a brain-computer interface. The neural interfaces may be used for recording and/or stimulation, e.g., for applying light, current, voltage, and/or drugs. Embodiments of the disclosed high-bandwidth neural interfaces may be deployed using minimally invasive surgical techniques that may reduce or eliminate collateral damage to normal brain tissue. The disclosed neural interfaces are advantageous over conventional devices and techniques, which may require highly invasive procedures and/or may result in damage to the areas of the brain surrounding the deployment zone.

High-bandwidth neural interfaces as described herein may also be capable of manipulation and/or adjustments to position during and after deployment within the nervous system. For example, the disclosed neural interfaces may include electrode arrays that are capable of being manipulated or repositioned one or more times after initial deployment in order to achieve optimal recording and/or stimulation performance in a dynamic, exploratory manner.

The disclosed high-bandwidth neural interfaces may also include electrode contacts configured to provide improved spatial and temporal resolution. The neural interface may be used for real-time, dynamic, three-dimensional electrophysiologic mapping of the brain. In some embodiments, the disclosed high-bandwidth neural interfaces may be used for mapping related to epileptic research and treatment. For example, high-bandwidth neural interfaces may be used for localization and stimulation of targets associated with temporal lobe epilepsy on the cortical surface and/or in deeper regions of the brain.

The disclosed high-bandwidth neural interfaces may include hundreds or thousands of electrode sites. Embodiments of the present disclosure may be manufactured using a microfabrication process that permits scaling to many tens of thousands of electrode sites.

Additionally, the neural interfaces may include integrated circuits on the implantable electrode array. While conventional arrays may not contain active electrodes, some embodiments of the present disclosure may include active electrodes on the implantable components.

High-bandwidth neural interfaces as described herein may be capable of being delivered via minimally invasive methods utilizing catheters and cannulas. For example, the present disclosure is directed towards neural interfaces that may be deployed to assume the shape of curved surfaces of the brain including but not limited to the outer cortical surfaces, the inner ventricular surfaces, and the inner surfaces of blood vessels within the brain. In some embodiments, the described neural interfaces are minimally invasive to the brain and may be used on the surface, e.g., conforming to curved brain surfaces and wrapping around the brain without penetrating brain tissue.

The neural interfaces described herein may be capable of delivery to the nervous system in a minimally invasive manner that may be well tolerated by a patient as compared to conventional delivery techniques. Furthermore, such delivery procedures may have improved safety, speed, and comfort. The delivery procedures may be less painful than conventional techniques and may reduce or eliminate the need for visible incisions.

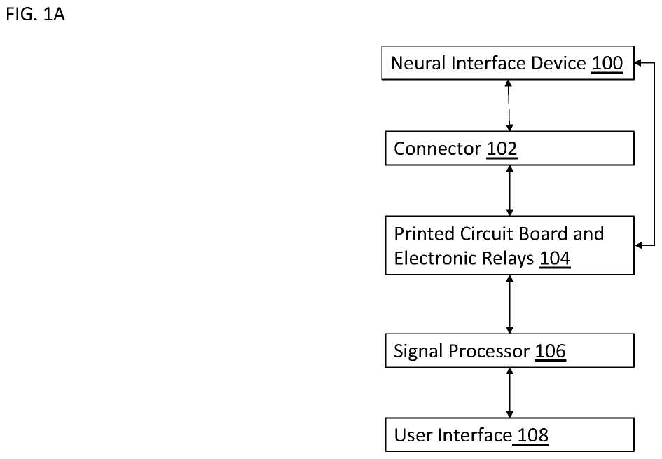

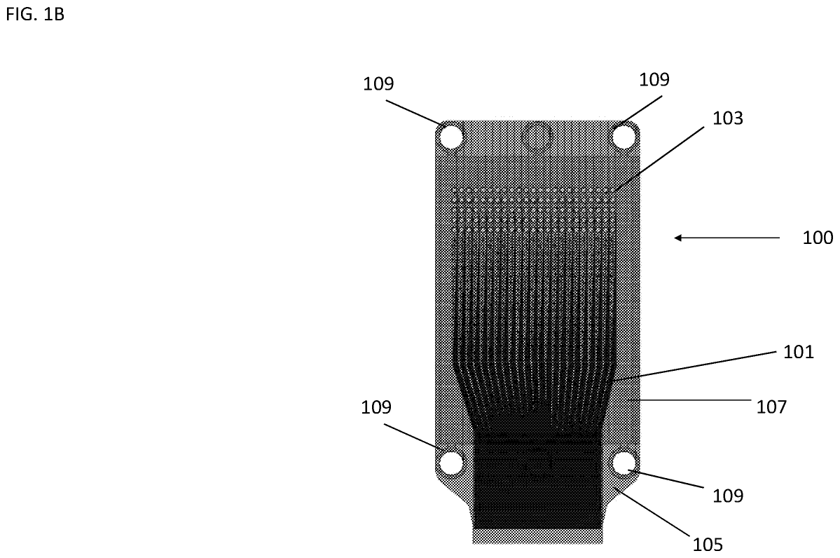

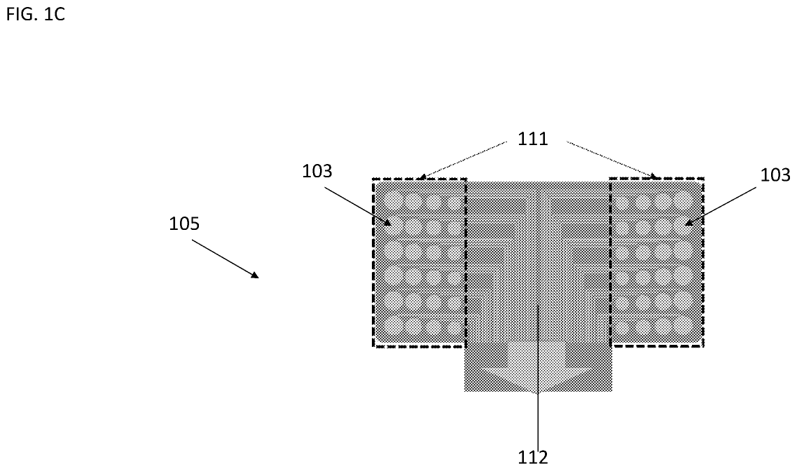

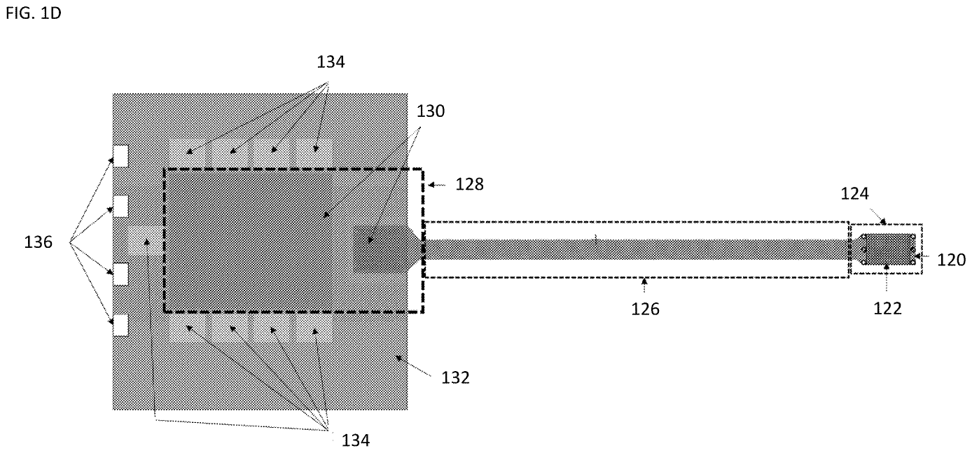

Referring now to FIG. 1A, a schematic diagram of a neural interface system is depicted in accordance with an embodiment. A neural interface device 100 may include electrode arrays configured for implantation at a target site of the brain and capable of recording or stimulating nervous tissue therein. The neural interface device 100 may be coupled to a connector 102 located outside of the skull by a cable or other interface. The cable may interface with a printed circuit board 104 (PCB) and/or other electronic signal relays. The PCB 104 may be configured to provide stimulation and/or recordation commands to the neural interface device 100. A signal processor 106 may be configured to receive signals from the PCB 104 and/or output signals to the PCB 104. The signal processor 106 may also be configured to generate a display of the recorded or stimulated neural signals on a user interface 108, which may be accessed on a laptop computer, a desktop computer, a mobile device, or another computer device as would be known to a person having an ordinary level of skill in the art. Digital input/output signal processing may occur between the user interface 108 and the signal processor 106 and/or between the signal processor 106 and the PCB 104. Further, one or more power sources (not shown) may be configured to provide power to any number of elements of the neural interface system including the neural interface device 100, connector 102, PCB 104, signal processor 106, and user interface 108. As will be discussed herein, the neural interface device 100 may include a microelectrode array that is connected via the connector 102 to the PCB 104. Electrophysiology chips may be bonded to the PCB 104 to enable communication with the signal processor 106 and/or the user interface 108 via digital input/output signals and power interfaces. In some embodiments, signals from the electrophysiology chips may be fed back to the neural interface device 100 or other effectors in a closed loop or other format.

In some embodiments the signal processor 106 may be configured to perform pre-processing, feature extraction, classification, and the like on signals acquired by the neural interface device 100 in connection with brain computer interface applications.

Referring now to FIG. 1B, an illustrative neural interface device 100 including a thin-film microelectrode array is depicted in accordance with an embodiment. The thin-film microelectrode array 105 may include a plurality of electrical traces 101 and electrodes 103 thereon. As shown, the thin-film microelectrode array 105 may be attached to a spring-like stent-mesh assembly 107 by way of eyelets 109. The illustrated embodiment of the neural interface device 100 may be deployable using a catheter or cannula. In some embodiments, the neural interface device 100 may include a microelectrode array 105 that is composed of a thin-film microelectrode having two lateral wing-like appendages located distal to a thin-film flexible cable that terminates at the proximal end in a thin-film connector region.

Referring now to FIG. 1C, an exemplary electrode array 105 with exaggerated wing features 111 is depicted in accordance with an embodiment. As shown, the electrode array 105 may include wing features 111 and a central region 112 therebetween that provides electrical connections between the electrodes 103 positioned on the wing features 111, thereby forming part of a cable that is configured to connect to a connector 102. In some embodiments, all electrodes 103 are disposed upon the wings 111. In additional embodiments, electrodes 103 are additionally or alternatively disposed upon the central region 112 of the electrode array 105.

The thin-film microelectrode array 105 may be fabricated using various microfabrication techniques. Exemplary, non-limiting techniques include spin-coating and curing of a polymer insulator base layer, lamination of a polymer insulator base layer, deposition and patterning of the electrode metal (i.e., trace metal, pad metal), spin coating and curing of a polymer insulator top layer, lamination of a polymer insulator top layer, patterning and/or etching of the top polymer layer to expose at least some of the pad and trace metal layer, and optional plating or deposition and patterning of a top pad or electrode material layer. The metals forming the electrodes 103 may include titanium, platinum, and/or gold in order to provide adhesion and/or conduction properties. Electrodes 103 may also be composed of organic conductive materials such as poly(3,4-ethylenedioxythiophene) polystyrene sulfonate (PEDOT:PSS) and/or inorganic conductive materials such as platinum, iridium and its compounds, ruthenium and its compounds, titanium nitride, and additional materials as would be apparent to a person having an ordinary level of skill in the art.

In some embodiments, the electrodes 103 comprise a polymer insulator which may include thermoplastics, thermoset polymers, liquid crystal polymers, semicrystalline polymers, and/or elastomers such as polyimide, polyetherimide (PEI), silicones, fluoroelastomers, polyurethanes, and Parylenes.

In some embodiments, the microelectrode array 105 may include an inorganic barrier or adhesion layer having silicon nitride and/or silicon carbide deposited following deposition of the first polymer base layer and the first metal trace and pad layer. In this manner, the trace and pad metal may be encapsulated with and in contact with an inorganic material in addition to the outer polymer layer encapsulation. In such embodiments, both the top polymer insulator layer and the inorganic barrier or adhesion layer may be patterned and etched either at the same time or in two separate patterning steps.

In some embodiments, the electrodes 103 of the microelectrode array 105 may be scaled from about 5 to about 500 microns in diameter and may be spaced by about 25 to about 1000 microns from center to center.

Referring now to FIG. 1D, a schematic view of an exemplary neural interface system is depicted in accordance with an embodiment. As shown, a neural interface device (e.g., the device 100) may include a winged thin-film microelectrode array 120 (e.g., the electrode array 105) provided at a distal end of the system. A stent-mesh 122 may be overlaid and attached to the winged thin-film microelectrode array 120 using eyelets. Together, the stent-mesh 122 and the winged thin-film microelectrode array 120 form a stent-mesh array assembly 124. As shown, the stent-mesh array assembly may be located at a distal end of a thin-film flexible cable 126 configured to connect at its proximal end to a connector assembly 128. The connector assembly 128 may include a proximal end of the microelectrode array including bonding and interconnection regions 130 disposed on the microelectrode array and interfacing printed circuit boards 132 (PCBs) as shown. In some embodiments, the interfacing PCBs 132 are composed of a single piece as shown. However, the interfacing PCBs 132 may be separated into multiple boards with different bond interfaces. Electrode interface integrated circuits 134 may be bonded to the interfacing PCBs 132 using, e.g., eutectic mixtures, thermocompression, and/or adhesive bonding. Furthermore, standard cable connectors may be used to communicate with other data collection devices. For example, cables may be attached to standard cable connectors 136 to transmit signals between the electrode interface integrated circuits 134 and external data collection and storage devices.

### Stent-Mesh System for Deployment of a High-Bandwidth Neural Interface

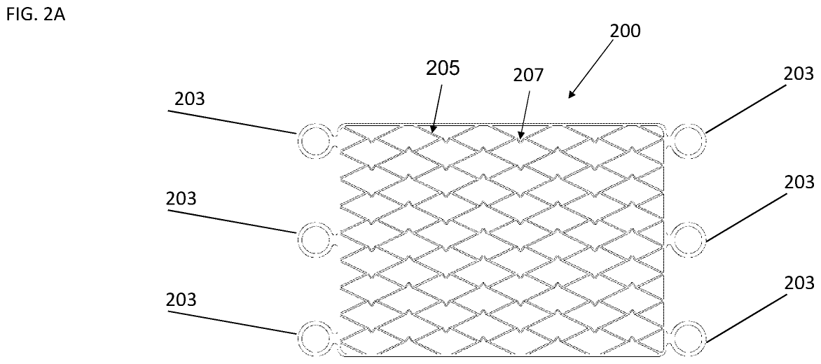

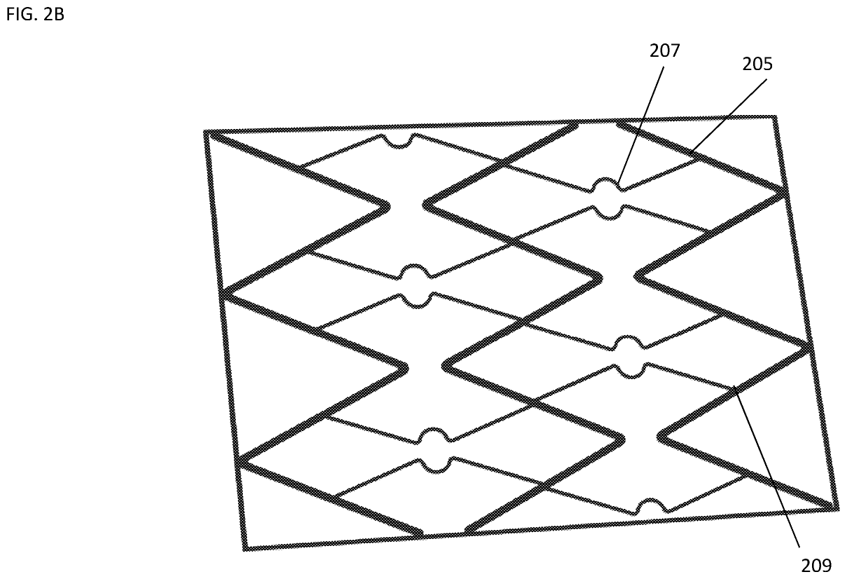

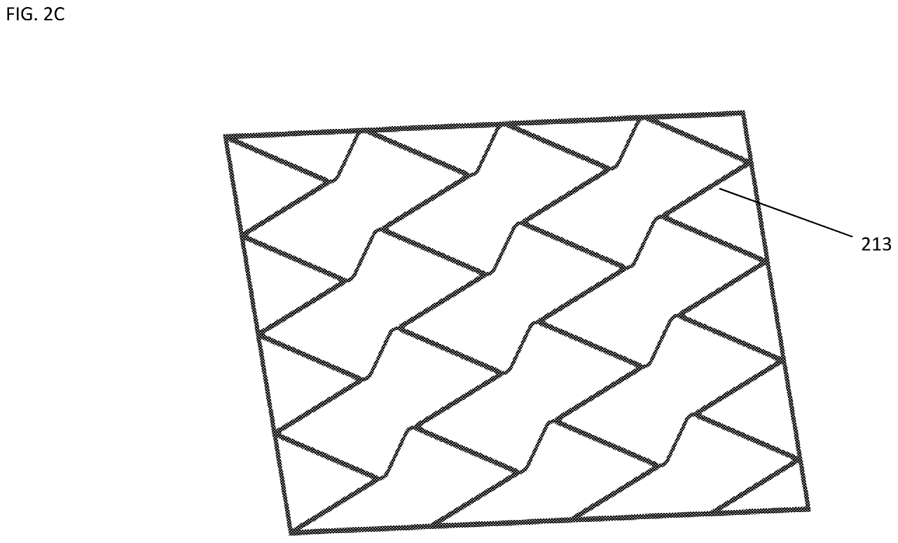

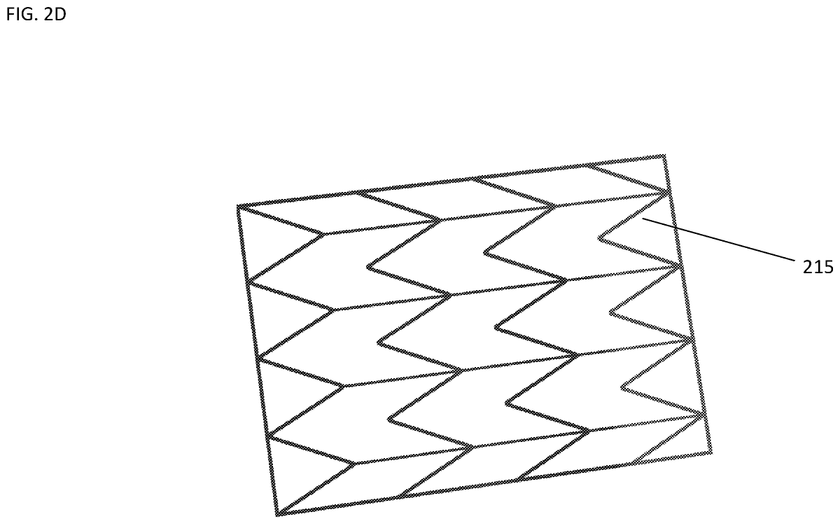

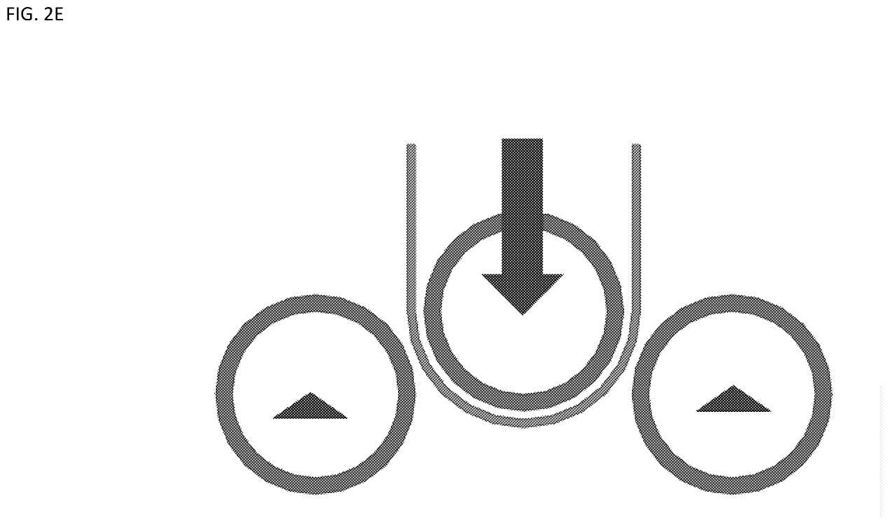

Turning now to FIGS. 2A-2E, various exemplary stent-mesh assemblies are depicted in accordance with an embodiment. The described and depicted stent-mesh assemblies 200 may be used as part of the stent-mesh assembly 107 of FIG. 1B and/or the stent-mesh array assembly 124 of FIG. 1D. In some embodiments, the stent-mesh 200 may be composed of nitinol and configured to have a substantially planar shape. In some embodiments, the stent-mesh 200 may be configured to be rolled-up or folded within a delivery system such as a catheter or cannula. In some embodiments, the stent-mesh 200 may be configured to return to a substantially planar shape capable of conforming to a target surface when deployed by the delivery system. In some embodiments, the stent-mesh 200 may be configured to regain its original shape after deployment. In some embodiments, the stent-mesh 200 may be configured to conform to a curved surface (e.g., a surface of brain tissue) without kinking.

The stent-mesh 200 may be configured to attach to an electrode array (e.g., microelectrode array 105 as described with respect to FIG. 1) such that the electrode array is configured to be positioned around a target area when the stent-mesh 200 is deployed from the delivery system. In some embodiments, the stent-mesh 200 may be attached to the electrode array via eyelets positioned along the perimeter of the stent-mesh 200. Stent-mesh 200 may additionally or alternatively be attached to an electrode array through mechanical or adhesive techniques as would be apparent to a person having an ordinary level of skill in the art. For example, at least a portion of the electrode array may be physically joined to at least a portion of the stent-mesh 200 such that the electrode array and the stent-mesh 200 retain individual and joint compliance required to fit into both confined spaces in a compressed state and/or expanded in a deployed state.

The stent-mesh 200 may be spring-like and have a structure or shape similar to a conventional stent that is cut along one line (e.g., along an axial length) to form a flat spring-like sheet. The stent-mesh 200 may be configured to provide mechanical support to the electrode array during rolling and/or insertion into confined spaces, e.g., within a lumen of a deployment catheter. The stent-mesh 200 may also be configured to provide mechanical support to the electrode array during deployment or retraction, i.e., while the stent-mesh-electrode array assembly is pushed or pulled from a deployment catheter. The stent-mesh 200 may have shape memory properties and/or elastic or resilient properties. For example, the stent-mesh 200 may be composed of shape memory or superelastic nitinol (i.e., nickel-titanium), shape memory polymers, or other polymers such as polyether ether ketone (PEEK) and PEI.

In some embodiments, a plurality of stent-meshes 200 may be associated with an electrode array. In some embodiments, a neural interface may include a plurality of electrode arrays associated with a single stent-mesh. In additional embodiments, a neural interface may include a plurality of electrode arrays, where each electrode array is associated with a plurality of stent-meshes. For example, multiple stent-meshes 200 with different dimensions may be deployed such that the total coverage area of the stent-meshes 200 is suitable to cover a substantial amount or the entirety of the cortical surface area, and/or a substantial amount or the entirety of a part of the inner surface of the brain, e.g., the ventricular surfaces. Furthermore, the dimensions of flexible cables associated with the electrode arrays may be varied to enable bundling a plurality of electrode arrays and/or tunneling of cables through a reduced number of exit pathways (e.g., a single exit pathway) in a given region.

In additional embodiments, a stent-mesh 200 may not be required for deployment and/or retraction of the electrode array. In such embodiments, the thickness, taper, width and/or length of the electrode array may be tailored with the elastic properties and restoring forces necessary to enable deployment and/or retraction without the assistance of a stent-mesh 200.

Referring now to FIG. 2A, the stent-mesh 200 may a mesh pattern that forms mesh openings 205 therein and eyelets 203 arranged about the mesh pattern. For example, the mesh pattern may be formed by strut elements 205 and ring elements 207 to create mesh openings therebetween. As depicted in FIG. 2A, the struts 205 and rings 207 may form a peak-to-peak bridge connection wherein the peaks of adjacent struts 205 are joined by the rings 207. In some embodiments, the features of the stent-mesh 200 may be composed of nitinol shape memory alloy, e.g., a nitinol sheet. In some embodiments, the nitinol material may have a thickness of about 0.002 inches. However, additional types and thicknesses of material are contemplated herein as would be apparent to a person having an ordinary level of skill in the art.

Eyelets 203 as depicted in FIG. 2A can be placed along the perimeter of the stent-mesh 200 and may function as attachment points between the stent-mesh 200 and the electrode array. In some embodiments, eyelets 203 may be positioned along a first dimension of the stent-mesh 200 having a narrower width. Eyelets 203 may be placed along the narrower dimension of the stent-mesh 200 such that they do not protrude from the perimeter of the stent-mesh 200 in a second dimension having a greater width. In this manner, the eyelets 203 may be placed so as to avoid interaction with the tissue and/or delivery device (e.g., a cannula) when the assembly is positioned, deployed, and the like. While the eyelets 203 are described as protruding eyelets herein, the eyelets 203 may additionally or alternatively comprise recessed eyelets.

FIG. 2B depicts an alternative design of a stent-mesh 200 in accordance with an embodiment. As shown, the stent-mesh 200 may include strut elements 205 and ring elements 207 that form a middle-to-middle bridge connection 209. In some embodiments, the mesh pattern may comprise repeating units of five struts 205 and three rings 207. In some embodiments, the mesh pattern may comprise repeating units of four struts 205 and four rings 207. In some embodiments, each strut 205 may have a length of about 3.5 mm. In some embodiments, each strut 205 may be composed of nitinol shape memory alloy having a thickness and width of about 0.002 inches.

FIG. 2C depicts a second alternative design of a stent-mesh 200 in accordance with an embodiment. As shown, the stent-mesh 200 may include strut elements 213 and connector or ring elements that form a shifted peak-to-peak bridge connection. In some embodiments, the mesh pattern may comprise repeating units of eight struts 213 and four rings. In some embodiments, each strut 213 may have a length of about 2.2 mm. In some embodiments, each strut 213 may be composed of nitinol shape memory alloy having a thickness of about 0.002 inches and width of about 0.003 inches.

FIG. 2D depicts a third alternative design of a stent-mesh 200 in accordance with an embodiment. As shown, the stent-mesh 200 may include strut elements 215 and connector or ring elements that form a peak-to-valley bridge connection. In some embodiments, the mesh pattern may comprise repeating units of eight struts 215 and four to five rings. In some embodiments, each 215 strut may have a length of about 2.2 mm. In some embodiments, each strut 215 may be composed of nitinol shape memory alloy having a thickness of about 0.002 inches and width of about 0.003 inches.

In the illustrated examples of stent architecture, the struts and openings depicted in the stent-meshes 200 may be configured to have dimensions between about 1 mm and about 3 mm in width and height. In some embodiments, the struts, rings, and openings may be designed to exhibit a specific bending stiffness in the length and width direction of the stent-mesh 200 based on desired properties. For example, the design in FIG. 2A may exhibit low bending stiffness in the first dimension and higher bending stiffness in the second dimension. In another example, the design in FIG. 2D may be stiffer in a first dimension (e.g., the longer dimension) than in a second dimension (e.g., the shorter dimension).

It should be understood that the examples of stent architecture depicted in FIGS. 2A-2D are for illustrative purposes. Various stent architectures may be used in connection with the disclosed systems and methods, including, but not limited to, braided, zig-zag, laser cut, and serpentine configurations.

FIG. 2E depicts a graphic representation of testing a stent architecture in accordance with an embodiment. For example, the stent architectures described herein may be tested using finite element analysis and/or direct mechanical flexural testing to target a designed pseudo-radial pressure, despite the final planar configuration. This pressure can be designed to assist in fixing the array in a desired folding geometry while inside the catheter or cannula during the deployment scheme, as well as to assist in counteracting restoring force during deployment from the catheter or cannula while the stent-mesh assembly is unfolding in the desired location and geometry.

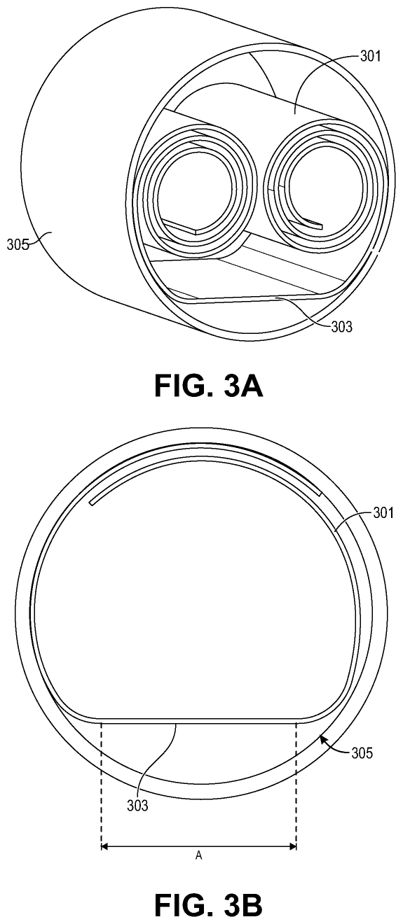

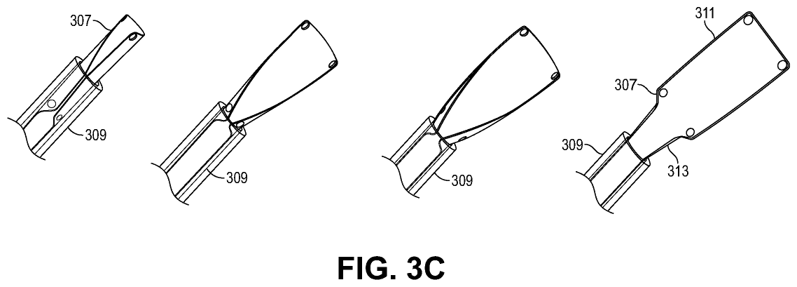

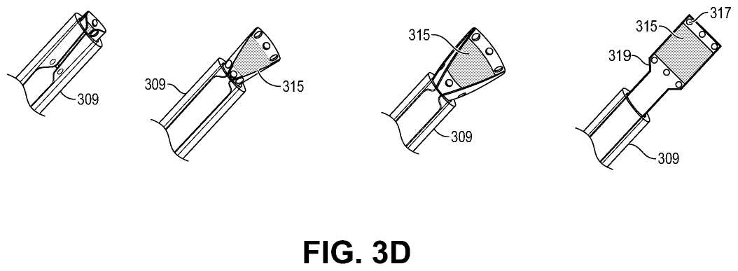

Referring now to FIGS. 3A-3D, an exemplary method for folding an electrode array and stent-mesh assembly is depicted in accordance with an embodiment. In some embodiments, a flex-cable may be one piece fabricated in the same process as a thin-film microelectrode array. In some embodiments, the microelectrodes in the array can be located at least partly on the wing appendages and/or at least partly on the central region of the electrode body coaxially with the flex-cable. The two lateral wings may be curled into a loose spiral to be inserted into a delivery catheter or another confined space, and the flex-cable and coaxial region of the microelectrode array may be flat or slightly curved in the same direction as the lateral wings for co-insertion in a delivery catheter or another confined space.

FIG. 3A provides a cross-sectional perspective view of a microelectrode array contained within a delivery catheter or confined space in accordance with an embodiment. As shown, the flexible cable may include wings 301 configured to curl into a loose spiral. The flexible cable may include a central region 303 configured to hold microelectronics. For example, the central region 303 may remain relatively unbent such that it may be positioned within the delivery catheter or confined space 305 without bending.

In some embodiments, as shown in the cross-sectional view of FIG. 3B, a width A of the central region 303 may be in the range of about 1.56 mm. Furthermore, the central region 303 may be retained in a relatively flat configuration. The curled or spiraled wings 301 may be configured to fold into the area substantially above the central region 303. The curled or spiraled wings 301 may be configured to fold within a catheter having an outer diameter of about 2.997 millimeters and an inner diameter of about 2.74 millimeters. However, alternative configurations and dimensions may be employed in order to suit a particular application as would be apparent to a person having an ordinary level of skill in the art.

In some embodiments, the neural interface may have flexible and/or rigid portions. The neural interface may require encapsulation in order to prevent interaction with biological tissue. For example, the neural interface may include one or more integrated circuits located within a central rigid region of the neural interface located between left and right wings of the microelectrode array. The integrated circuit chip encapsulation may be composed of underfill that is coated in a patterned barrier layer that is configured to prevent corrosion of the integrated circuits and their packaging. In some embodiments, the patterned barrier layer may include dielectric barriers deposited by atomic layer, chemical, and/or physical vapor deposition, with or without plasma assistance. In some embodiments, the patterning may be applied by shadow-masking, selective lift-off of the barrier using a patterned sacrificial material, and/or patterning of a mask material followed by etching.

In some embodiments, the configuration and dimensions of the thin-film microelectrode array and stent-mesh may be configured for insertion into the confined space or catheter with a minimum bend radius selected and defined such that no component of the array-mesh assembly exceeds a predefined strain limit.

In some embodiments, the array-mesh assembly may be inserted or loaded into a catheter, e.g., the array-mesh assembly may be rolled up as illustrated in FIGS. 3A-3B and further described with respect to FIGS. 3C-3D herein. Accordingly, the array-mesh can be advanced to a target area and then deployed from the catheter as depicted in FIGS. 3C-3D.

Various techniques for loading the array-mesh assembly into the catheter or delivery device are envisioned. For example, one method of loading the mesh-array assembly into the delivery and/or deployment catheter or other desired confined space includes fixing one side of the mesh to a mandrel or other shape template such that the mesh-array assembly can be rolled by hand and/or with the assistance of a tool into the desired shape around the shape template followed by retraction of the shape template.

An alternative method for loading the mesh-array assembly into the delivery and/or deployment catheter or other confined space includes cooling of the mesh-array assembly below a phase transformation temperature of the shape memory material that the mesh is composed of, such that the material transitions into a state where it can be easily deformed. Cooling can be achieved using a cold bath or flow of coolant over the mesh-array assembly, and deformation can occur up to the strain limit of the material and so that the material maintains shape memory when brought above the activation temperature.

Another alternative method of loading the mesh-array assembly into the delivery and/or deployment catheter or other confined space includes slight pre-bending of the mesh-array followed by insertion into a stent crimping device. The stent crimping device lumen can then be closed mechanically with electrical or mechanical assistance. The stent crimping device may be used to compress the appendages of the mesh-array radially inward, causing them to curl along the walls of the crimping device lumen. A fixed or movable shape template can be used to fix the portion of the mesh-array coaxially with the flex-cable assembly in place during closing of the crimping device, such that the appendages of the mesh-array curl into the desired configuration and dimensions while some portion of the mesh-array remains in a different desired shape. The inside of the stent crimping device or the outside of the mesh-array can be fixed or coated with a lubricious material to reduce friction between the mesh-array and the device during curling of the mesh-array.

FIG. 3C depicts the deployment or unfurlment of an electrode array in accordance with an embodiment. In particular, the curled distal end of the array 307 unfurls upon deployment from the cannula 309 to its final expanded state 311. In some embodiments, the electrode array 307 may be configured to unfurl based on its own physical properties. For example, the electrode array 307 may be capable of unfurling from a curled state in the cannula 309 to a flat deployed state 311 using a spring-like return due to the inherent resilience and/or elasticity of the electrode array 307. In such an embodiment, the electrode array may include features such as tapers 313 that may be used to guide the unfurling process or guide curling on retraction along these features.

FIG. 3D depicts the deployment or unfurlment of an electrode array and stent-mesh assembly 315 in accordance with an embodiment. As illustrated, the electrode array may be attached to the stent-mesh by way of the eyelets 317 to form the assembly 315. Together, the entire assembly 315 may be unfurled. Unfurling or deployment may be guided by features such as tapers 317.

Tapers as depicted and described herein may be provided on a proximal end of the assembly to encourage unfolding or unfurling and/or to encourage re-folding or re-furling when an assembly and/or the stent-mesh component thereof are pulled proximally to be returned within a deployment device (e.g., a catheter). In some embodiments, tapers as depicted and described herein may additionally be provided on the distal end of the assembly to assist with insertion or movement through a confined space (e.g., through a tissue or vessel) and/or loading into a folded state within the deployment device.

In some embodiments, the neural interface device may be configured to deploy the thin-film microelectrode array from the catheter by deploying the stent-mesh assembly connected to the thin-film microelectrode array. Then, the stent-mesh may be removed from the thin-film microelectrode array and then retracted and removed via the catheter.

For example, when deployed from a delivery catheter, the mesh-array assembly including the thin-film microelectrode array attached to the stent-mesh, can be pushed out of the confined space of the delivery catheter. A force may be applied upon the proximal end of the mesh via the guidewire. As the mesh is pushed out of the confined space of the delivery catheter, the mesh may be configured to expand to its original flat configuration due to spring force and/or shape memory recovery. Subsequently, the microelectrode array connected to the mesh may be configured to unfurl. In particular, the lateral wings of the microelectrode array may unfurl from their curled configuration as the lateral edges of the mesh expand to a flat configuration.

Once the thin-film microelectrode array is appropriately positioned, the stent-mesh may be removed from the thin-film microelectrode array. Various methods may be used to detach the stent-mesh from the thin-film microelectrode array depending on the mechanism by which the stent-mesh was attached to the thin-film microelectrode array. For example, the stent-mesh can be removed by the microelectrode array using a mechanical mechanism which manipulates the mesh using a guidewire that is actuated to move mechanical attachment features such as hooks out of corresponding attachment points on the array.

In some embodiments, the stent-mesh may be attached to the microelectrode array via a suture or other mechanical attachment. Accordingly, the stent-mesh may be removed by utilizing the properties of a bioresorbable material used to form the suture or mechanical attachment. Examples of bioresorbable materials include PGA filament, PEG adhesive, or other materials that are configured to dissolve or reduce in strength upon insertion in a fluid containing chamber in the body. These properties of the sutures may weaken the material such that mechanical release of the sutures from the mesh or array is enabled with a substantially low force such as pulling by a hook or other external utilized on the end of a catheter.

In some embodiments, the stent-mesh can be retracted by mechanical retraction of the mesh once removed from the array. In some embodiments, a stiff structure in the shape of a triangle or cone may be affixed to the proximal end of the mesh and to the wire assembly, which causes curling of the mesh back into its spiral shape when pulled back into the delivery catheter.

In some embodiments, the deployment and retraction of the microelectrode array may utilize a two-way shape memory effect for the stent-mesh assembly. For example, the stent-mesh may be brought above an activation temperature within the body for deployment. Later, the stent-mesh may be transitioned to below the activation temperature such that it can be retracted from the tissue surface using external retraction methods. Examples of external retraction methods may include use of a cooling device brought above the surface of the tissue configured to flow cooling solution through, alongside, or on the mesh surface. External retraction methods may also include a local cooling device deployed inside of or from a catheter or from outside the body. Examples of local cooling devices include Peltier or other thermoelectric devices.

In some embodiments, the deployment of the microelectrode array may involve a composite mesh that is composed of a polymer support with embedded superferromagnetic nanoparticles. In some embodiments, the nanoparticles may be used to deploy the microelectrode array in response to an applied external magnetic stimulus. The composite material may also be formed from a continuous sheet attached to the backside of another mesh.

In some embodiments, the stent-mesh assembly may be fabricated to have shape memory or have superelastic properties. For example, fabrication of the stent-mesh assembly may involve the placement of mesh between one or more insulator layers and dielectric layers, which may be configured to form the fabrication substrate for the array. Placement of the mesh between the insulator and/or dielectric layers may include the steps of thin-film deposition, followed by patterning on a polymer insulator base layer, followed by a deposition of a polymer top layer. The resulting structure may include a sandwiched structure having thermal lamination of a mesh between thermoplastic insulator layers, or adhesive lamination of a mesh between polymer insulator layers.

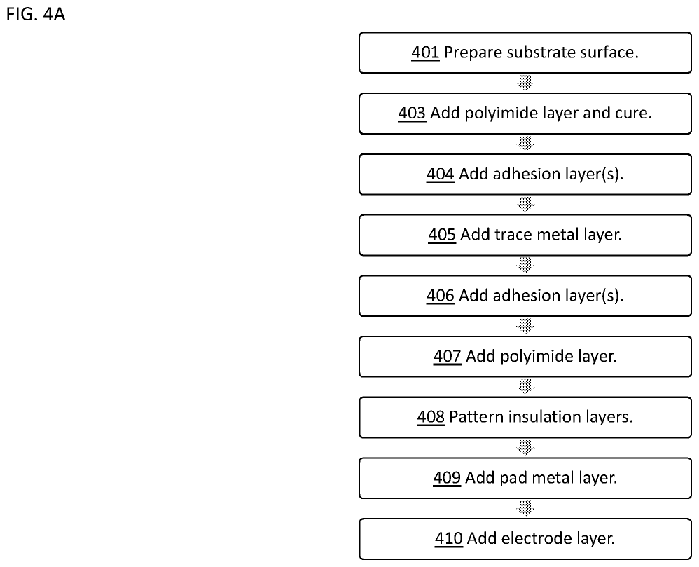

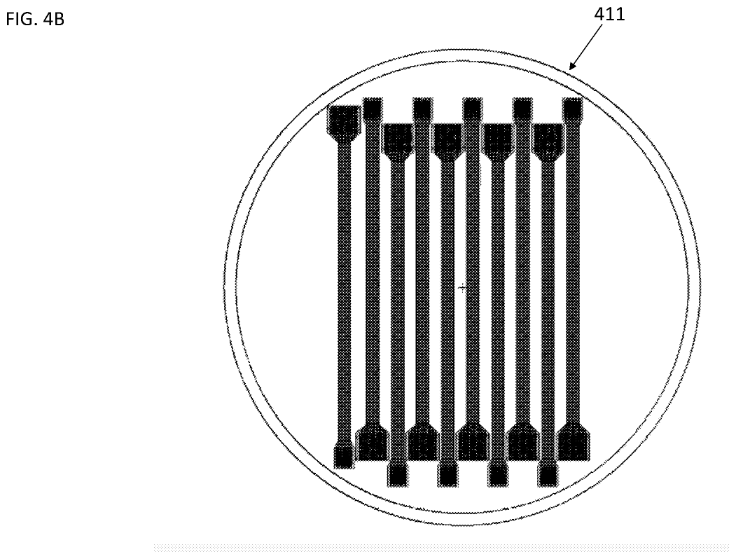

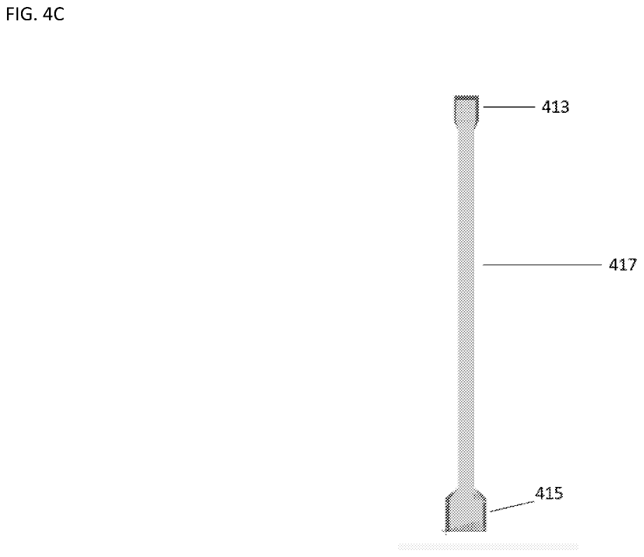

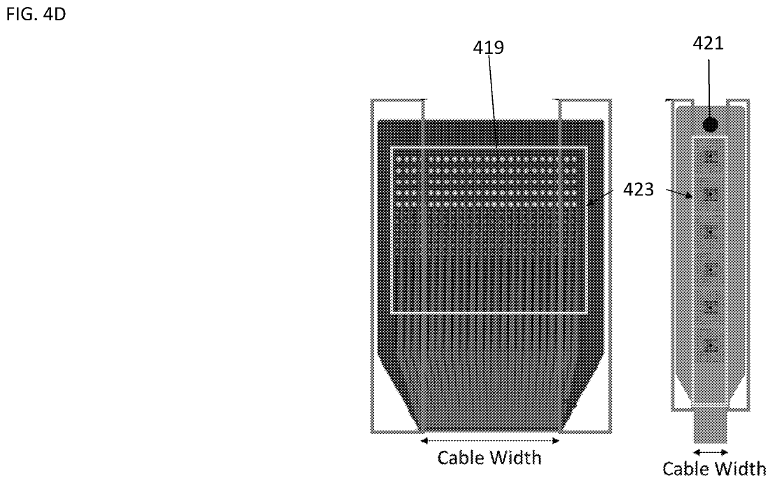

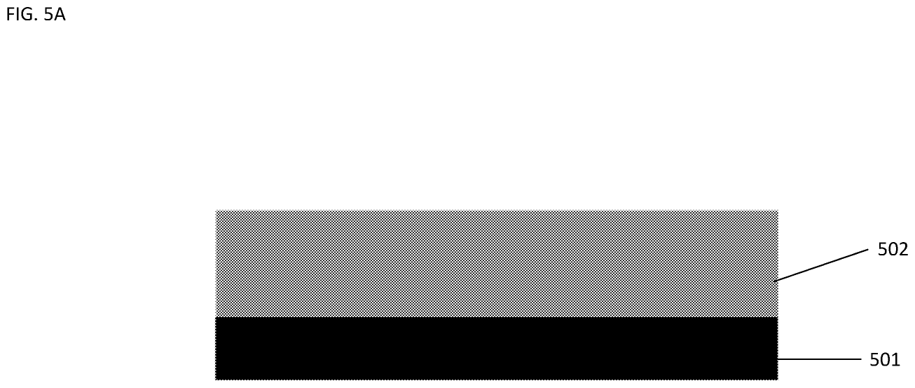

Referring now to FIGS. 4A-4D, a process for manufacturing the microelectrode array is depicted in accordance with an embodiment. In some embodiments, the microelectrode array described herein may include a stack composed of polyimide, trace metal, polyimide, and pad metal layers, respectively. In some embodiments, each of the polyimide layers may have a thickness between about 9-12.5 microns. In some embodiments, the metal layers, including the trace metal and pad metal layer, may each have a thickness of approximately 500 nm. In some embodiments, the microelectrode array may be approximately 14 mm at its widest section and approximately 150 mm in length. In some embodiments, a 200 mm wafer process with a 5 mm edge exclusion may be used to manufacture the microelectrode array. In some embodiments, a large area glass panel can be used as the device substrate using alternate glass coating processes such as dip or blade coating instead of spin-coating of polyimide and/or photoresist layers described below.

FIG. 4A depicts a flow diagram of an exemplary method for fabricating a microelectrode array in accordance with an embodiment. The method may be used to fabricate multiple electrodes. As illustrated in FIG. 4A, the process may include the steps of preparing a substrate surface 401, creating a first biocompatible polyimide layer and curing 403, optionally adding adhesion layers 404, patterning a trace metal layer 405, optionally adding adhesion layers 406, depositing a second polyimide layer 407, patterning an insulation layer 408, creating a pad metal layer 409, and creating an electrode layer 410.

In contrast to conventional methods, the disclosed manufacturing method may include lamination as an option. Additionally, while conventional methods specifically add adhesion layers like ceramics, the disclosed methods may utilize chemical treatments for creating an adhesive layer, for example in step 404. Further, in an embodiment of the disclosed neural interface having active electronics layers, the specific inclusion of additional components is balanced with a need for flexibility such that the microelectrode array retains its ability to fold.

In some embodiments, the step of preparing a substrate surface 401 may include requisitioning a silicon wafer or large area glass substrate at least about 8 inches in diameter and dehydrating the wafer using a baking process, plasma treatment of the substrate surface, or chemical treatment of the substrate surface. Dehydration of the substrate can occur on a hot plate or in an oven at a temperature above the boiling point of water, in vacuum or in atmospheric air. Plasma treatment of the substrate surface can include but is not limited to directional or isotropic plasma of oxygen, argon, nitrogen, and fluorinated or non-fluorinated hydrocarbons. Chemical treatment of the substrate surface can include but is not limited to dipping in acids or bases, and/or treatment or coating of a chemical primer.

In a second step 403, a first polyimide layer may be added and cured. Adding the first polyimide layer may include steps such as spinning, dip, or blade coating a polyimide precursor, followed by a soft bake process and curing. For example, the first polyimide layer may have a target thickness between about 9 and about 12.5 microns. In some embodiments, the polyimide may include a 3,4,3′,4′-biphenyltetracarboxylic dianhydride—p-phenylene diamine (BPDA-PPD) polyimide formed from a BPDA-PPD polyamic acid liquid resin. In some embodiments, the polyimide may include a BPDA-PPD polyimide laminated with or without a polyimide-based adhesive. The polyimide may be cured with exposure to nitrogen at 350 degrees Celsius for approximately 30 minutes. Alternative curing techniques such as vacuum curing or other temperature and timing conditions may also be used. Although polyimide is discussed herein, alternative biocompatible insulators may be used including, for example, epoxy, silicone, polyparaxylene, parylenes, and/or acrylic. The biocompatible layer may be configured to be implantable for long periods of time in biological tissue without inciting an immune reaction.

In a third step 404, insulating adhesion layers can be introduced using chemical processing, including but not limited to, acid or base processing or chemical priming. Adhesion layers can alternately be introduced by chemical, atomic, or physical vapor deposition of ceramic materials, including but not limited to, silicon oxide, silicon nitride, silicon oxynitride, silicon carbide, silicon oxycarbide, and transition metal oxides.

In a fourth step 405, a trace metal layer may be added onto the first biocompatible polyimide layer using a photolithography process followed by metal deposition. Examples of metals that may be deposited include titanium, platinum, gold, and the like. Metal layers may have a thickness of between about 20-500 nm.

A photolithography process for trace metal patterning can include the steps of spin-coating of a layer of photoresist, soft baking the photoresist layer, exposing the layer of photoresist using light in the pattern of the metal traces, post-exposure baking and development of the photoresist, plasma de-scumming of the patterned photoresist, deposition of metal using physical vapor deposition, lift-off of metal and photoresist using a solvent bath, and de-salting of the surface including patterned metal. An alternative process can include the steps of physical vapor deposition of metal, spin-coating a layer of photoresist, exposing the layer of photoresist using light in the pattern of the metal traces, post-exposure baking and development of the photoresist; plasma de-scumming of the patterned photoresist, etching of the metal using plasma or wet chemical etching, and stripping of photoresist using surface plasma de-scumming followed by solvent stripping.

In a fifth step 406, a second set of insulating adhesion layer(s) can be introduced using chemical processing, including, but not limited to, acid or base processing or chemical priming. Adhesion layers can alternately be introduced by chemical, atomic, or physical vapor deposition of ceramic materials including, but not limited to silicon oxide, silicon nitride, silicon oxynitride, silicon carbide, silicon oxycarbide, and transition metal oxides.

In a sixth step 407, a second insulation layer (e.g., polyimide) may be applied using spin coating techniques, soft baking the surface on a hot plate, and curing the surface at low temperatures. For example, the second insulation layer may include polyimide at a thickness of between about 9 to about 12.5 microns.

In a seventh step 408, all insulating layers can be patterned using photolithography and etching. A photolithography process for etching insulation layers can include deposition of a hard mask material such as metal or ceramic, spin-coating and soft baking of a photoresist layer, light exposure of the photoresist layer, post-exposure baking and development of the photoresist layer, etching of the hard mask material, and subsequent etching of the insulation layers using appropriate plasma or wet chemistries for each layer. Insulation layer patterning may be performed in one or multiple photolithography and etching steps, and chemistries may include acid or base etching solutions, or plasma chemistries including oxygen, hydrogen, fluorocarbon, hydrocarbon, and/or inert gases.

In an eighth step 409, a second metal layer, a pad metal layer may be formed using lift-off photolithography similar to trace metal layer 405. Metals may be deposited onto the surface by electron beam evaporation after which a solvent bath may be used for performing metal lift-off on the surface. The second metal layer may be composed of at least one of titanium, platinum, and gold having a depth of about 20-500 nm.

In a ninth step 410, an electrode layer can be added using electrodeposition, or it can be added using lift-off photolithography similar to pad metal layer 409. The electrode layer may be composed of at least one of titanium and its compounds, platinum, gold, iridium and its compounds, ruthenium and its compounds, having a total thickness of about 100 to about 1000 nm.

Electrical traces built in accordance with the methods described herein may require specific materials, thickness, width, and length dimensions to maintain an electrical pathway that is much lower in impedance than the electrodes under intended measurement conditions. Electrical traces are specified in spacing to minimize cross-talk between channels. Electrical trace patterns can include smooth bending curvature in fan-out and other regions to minimize local stresses in flexure.

Electrode array patterns can include exposed electrode surfaces from 10 to 2000 micron in diameter and can be placed at a center-to-center pitch of 25 to 3000 micron. Electrode surfaces can be circular in shape or defined in an arbitrary shape such as a regular polygon.

FIG. 4B provides an illustration of an example layout for a wafer having a plurality of microelectrode arrays in accordance with an embodiment. A wafer 411 is illustrated with a 200 mm area and 5 mm edge exclusion.

FIG. 4C provides an example of a microelectrode array in accordance with an embodiment. As illustrated the array 417 may include a distal end 413 and a proximal end 415. In one example, the distal end 413 may include a first polyimide layer having a thickness of approximately 12.5 microns. The first metal layer may be composed of titanium, platinum, gold, platinum and titanium at a thickness of about 20, 20, 500, 20, and 20 nm, respectively. A second polyimide layer may also have a thickness of approximately 12.5 microns. The electrode opening diameter, or etched surface may have a diameter between about 10-2000 microns with an electrode opening pitch of between about 25-3000 microns. The second metal layer may have an electrode edge overlap of about 5 to 25 microns. The trace width may be configured to be about 5 microns, and lines may be spaced about 5 microns apart. Additional insulating and/or adhesion layers may be included between metal and polyimide layers as described above.

As illustrated the array 417 may include a distal end 413 and a proximal end 415. The proximal end 415 may include a first polyimide layer having a thickness of approximately 12.5 microns. The first metal layer of the proximal end 415 may be composed of titanium, platinum, gold, platinum and titanium at a thickness of about 20, 20, 500, 20, and 20 nm, respectively. A second polyimide layer of the proximal end 415 may also have a thickness of approximately 12.5 microns. A metal pad of the proximal end 415 may be formed having a thickness of approximately 500 nm. The metal pad may have an opening diameter of 10-230 microns, with a pitch of 40-500 microns. The electrode edge of the metal pad of the proximal end 415 may be configured to overlap at about 10 to 25 microns. The trace width may be configured to be about 5 microns, and lines may be spaced about 5 microns apart. Additional insulating and/or adhesion layers may be included between trace metal and polyimide layers of the proximal end 415 as described above.

In one example, the distal end 413 of the array 417 may include a first polyimide layer having a thickness of approximately 12.5 microns. The first metal layer of the distal end 413 may be composed of titanium, platinum, gold, platinum and titanium at a thickness of 20, 20, 500, 20 and 20 nm, respectively. A second polyimide layer of the distal end 413 may also have a thickness of approximately 12.5 microns. The electrode opening diameter, or etched surface of the distal end 413 may have a diameter between about 10-2000 microns with an electrode opening pitch of between 25-3000 microns. The second metal layer of the distal end may have an electrode edge overlap of about 5 microns. The trace width may be configured to be about 5 microns, and lines may be spaced about 5 microns apart.

In some embodiments, as illustrated in FIG. 4D, the microelectrode array may include a fan-out region in one or both of the distal end 421 and the proximal end 419. In some embodiments, the distal end 421 may have a width and height of approximately 8500 by 11630 microns including the fan-out region. The distal fan-out length may be approximately 2800 microns and form an approximately 29 degree angle from the vertical. In some embodiments, the proximal end 419 may have a width and height of approximately 14020 by 15450 microns including the fanout region. The proximal fan-out length may be approximately 3849 microns and form an approximately 39 degree angle from the vertical. The total device thickness may be approximately 26 microns at the thickest cross-section of microelectrode array. The overall minimum trace width and inter-trace space may be approximately 1 to 5 microns. The overall device length may be approximately 15 to 20 centimeters.

The described fan-out or fan-in may allow the metal traces to expand from the minimum trace width and inter-trace space to the specified larger or smaller trace space or width in the proximal or distal ends. The described fan-out or fan-in may include a taper in the insulation geometry that traverses the differential width between the cable and the proximal or distal ends. The wings described at the distal end of the array may be composed of the regions of the film stack that extend beyond the edges of the cable width, and the taper in the fan-out or fan-in region also contributes to the folding behavior of the wings in a given array deployment scheme. For example, the taper may be configured to encourage the wings to unfold smoothly and uniformly on either side as the termination of the cannula lumen traverses the taper from its widest to its narrowest point during pushing of the array out of the cannula. Conversely, if the array is pulled back into a cannula for retrieval, the wings can be encouraged to fold smoothly and uniformly on either side back into a uniform coil as the cannula lumen traverses the taper from its narrowest to its widest point. As described herein, it should be understood that tapers may be included on the proximal and/or distal regions of the microelectrode array to encourage unfolding or unfurling, to encourage re-folding or re-furling, to assist with insertion or movement through a confined space (e.g., through a tissue or vessel), and/or to assist with loading into a folded state within the deployment device.

As illustrated in FIG. 4D, the electrode array patterns 423 along the fan out regions can be arranged in regular size and pitch or can include variations in pitch to effect resolution variation within a cluster of electrodes. For example, a given array can have 4 to 8 subarrays of electrodes disposed along a strip or grid, with each subarray having pitch of 400 to 200 micron at 0 to 1000 micron from the center of the subarray, pitch of 200 to 400 micron from 1000 to 2500 micron at 1000 to 2500 micron from the center of the subarray, and so on to the terminal radius of a given subarray. In the example subarray with outer edge electrodes terminating at 2500 micron from the center of the subarray, the electrode array may be composed of 4 to 8 subarrays with center-to-center subarray pitch of 5500 to 10000 micron. Pitch, size, and regions where various densities of electrodes are disposed can be optimized on a given device to match the physiology of a region of interest or for spatial resolution, while spacing of subarrays must be sufficient to provide trace routing along the body of the distal end of the overall array.

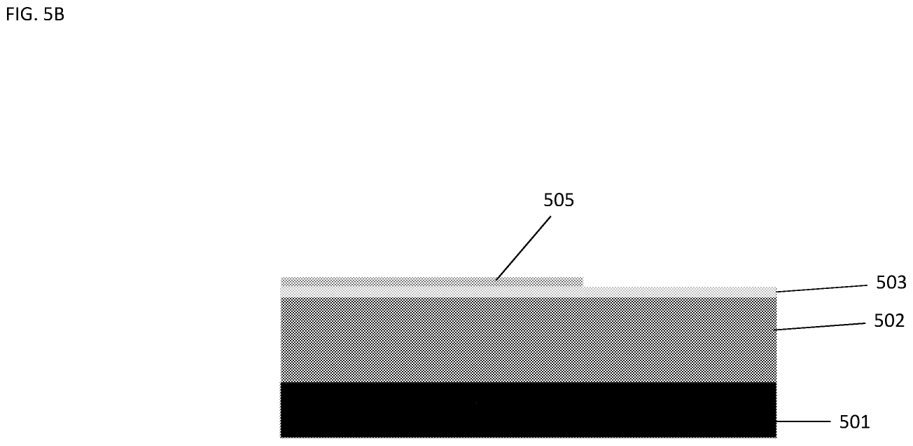

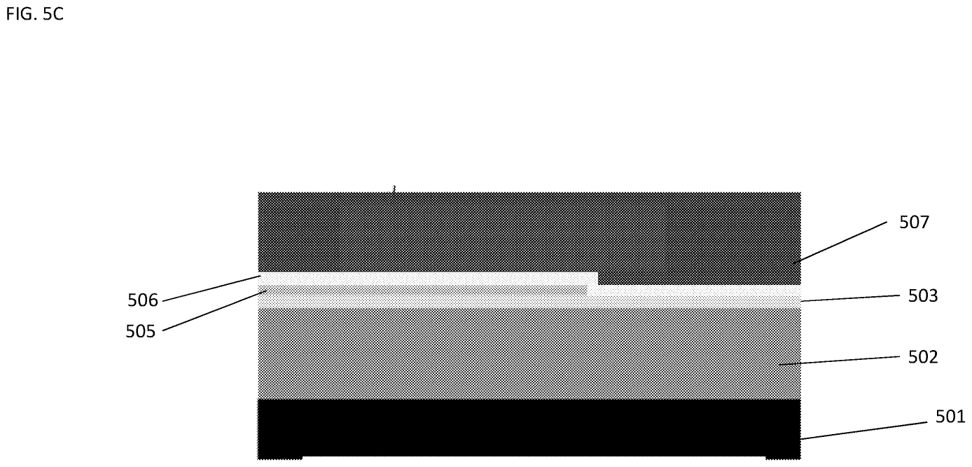

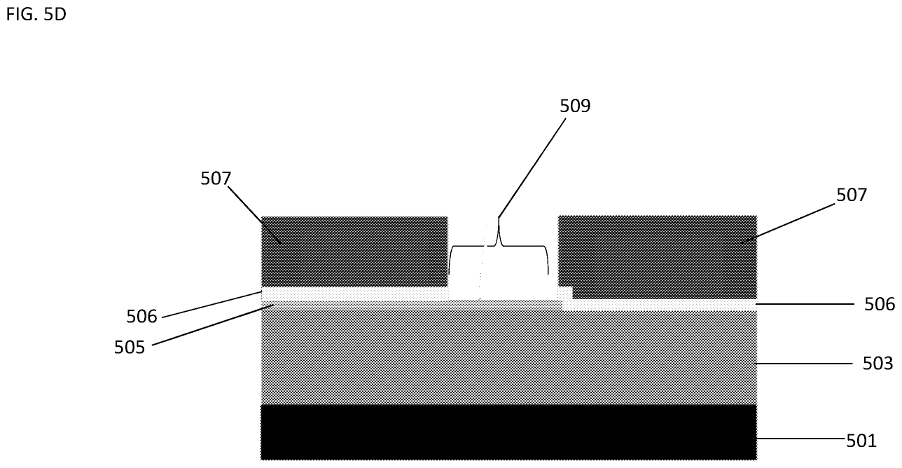

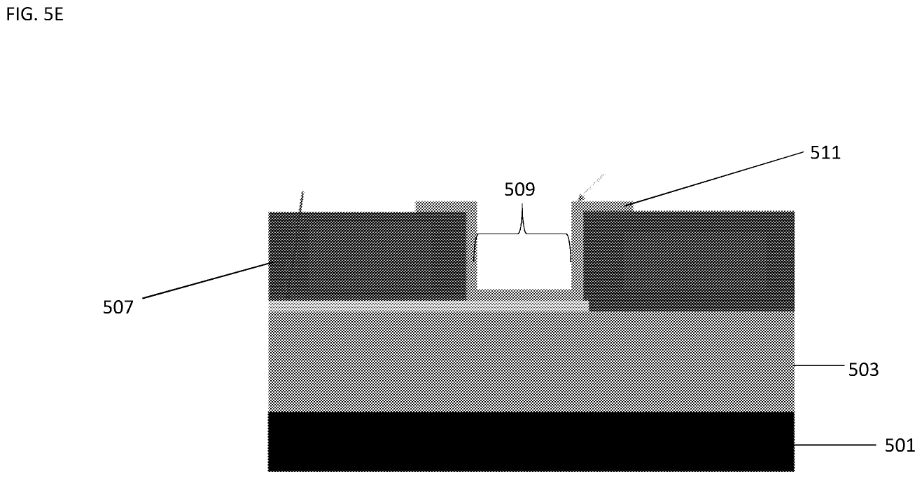

FIGS. 5A-5E illustrate a process for manufacturing the microelectrode array in accordance with an embodiment. As illustrated in FIG. 5A, in a first step of the manufacturing process, a first insulator layer 502 is spun onto a silicon substrate 501 and cured. In some embodiments the first layer 502 includes polyimide at a thickness of approximately 12.5 microns.

As illustrated in FIG. 5B, in a second step of the manufacturing process, a metal layer 505 is added to the cross-section. The metal layer 505 may have a thickness of about 580 nm and may be added using techniques of photolithography, deposition, and liftoff. In some embodiments, an insulating adhesive layer 503 can be introduced using chemical treatments or a deposition process.

As illustrated in FIG. 5C, in a third step of the manufacturing process a second insulator layer 507 is spun onto the cross-section and cured. The second insulator layer 507 may also be composed of polyimide having a thickness of about 12.5 microns. In some embodiments an insulating adhesive layer 506 can be introduced using chemical treatments or a deposition process.

As illustrated in FIG. 5D, in a fourth step of the manufacturing process, photolithography and/or etching techniques may be used to create an exposed region 509 of the trace metal layer 505.

As illustrated in FIG. 5E, in a fifth step of the manufacturing process, a second metal 511 may be added to the cross-section using techniques in photolithography, deposition, and liftoff. The second metal 511 may form a pad metal having a total thickness of about 580 nm.

In some embodiments, additional insulator layers composed of silicon carbide or nitride or the like may be used between polymer layers and trace metals.

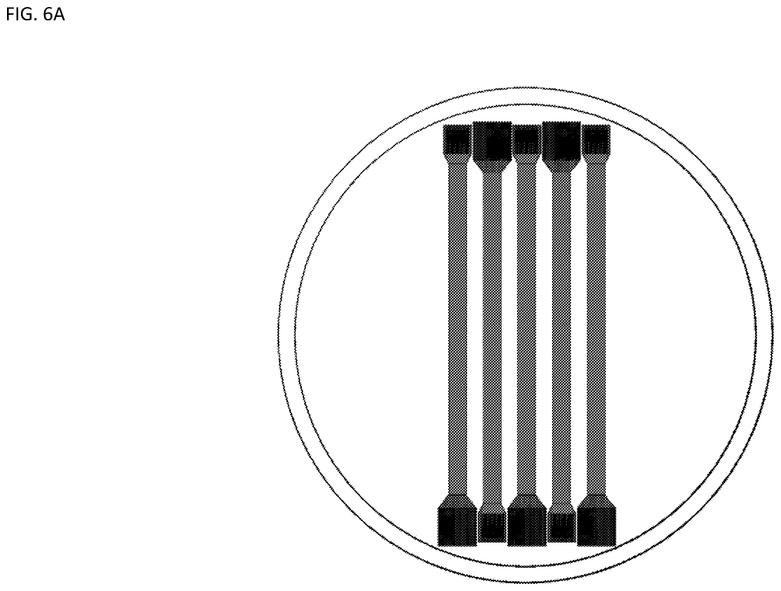

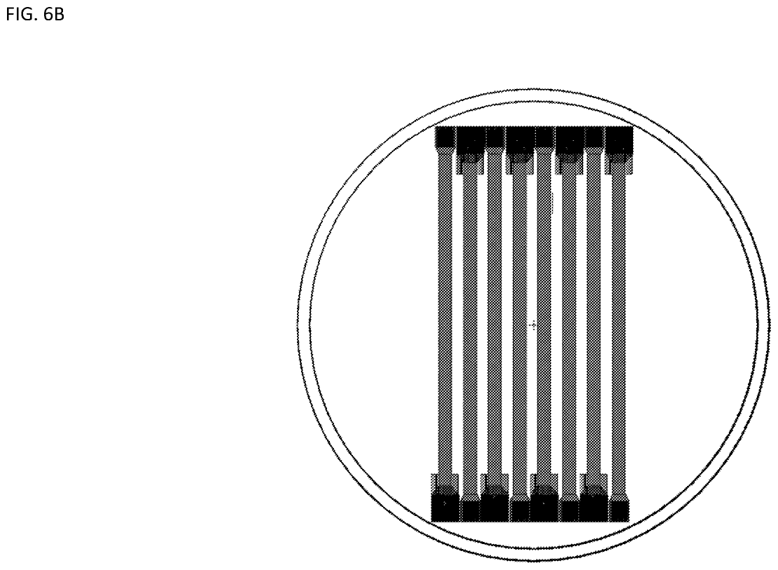

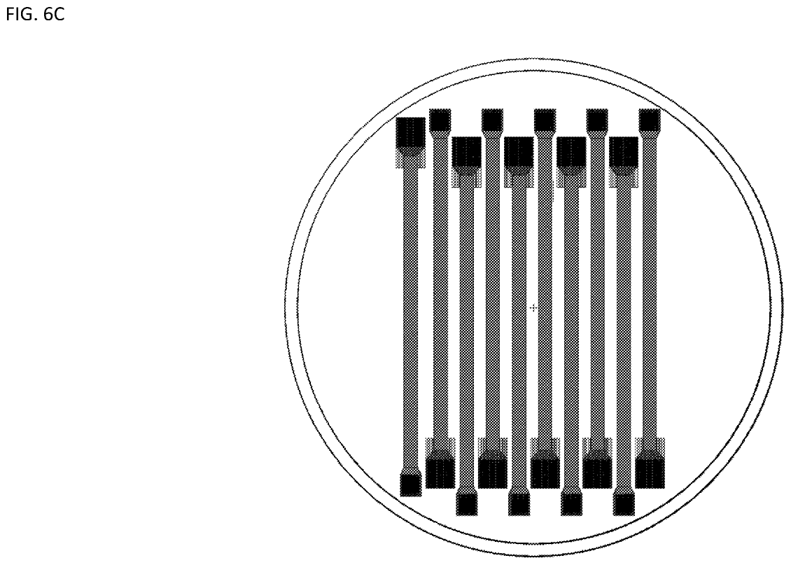

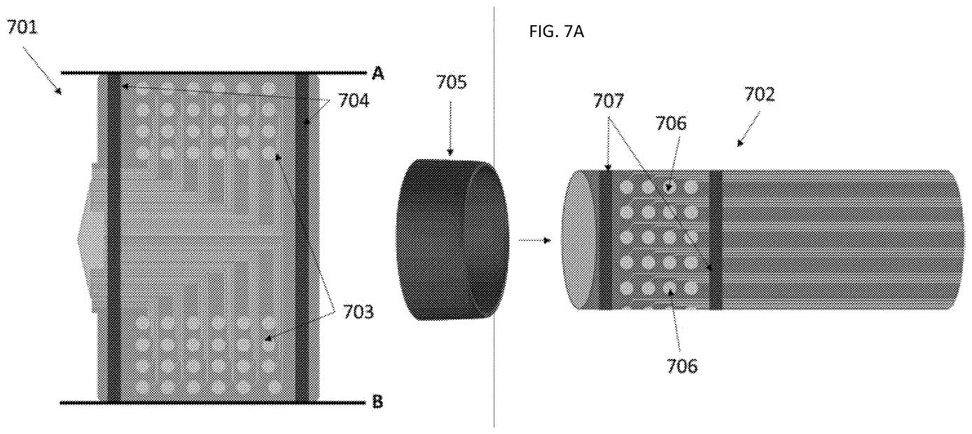

FIGS. 6A-6C illustrate examples of wafer design in accordance with various embodiments. FIG. 6A provides an example 6″ wafer layout for thin-film electrode array fabrication in accordance with the systems and methods described herein, with an approximate cable length of 100 mm. In particular, with this cable length and wafer size, only 5 devices of approximately 13 cm in length can be disposed on a wafer in a tight formation.

FIG. 6B provides an example 8″ wafer layout for thin-film electrode array fabrication in accordance with the systems and methods described herein, with an approximate cable length of 125 mm. In particular, with this cable length and wafer size, up to 8 devices of approximately 150 mm in length can be disposed on a wafer in a tight formation as shown.

FIG. 6C provides an example wafer layout for thin-film electrode array fabrication in accordance with the systems and methods described herein, with an approximate cable length of 125 mm. In particular, with this cable length and wafer size, up to 10 devices of approximately 150 mm in length can be disposed on a wafer in an offset formation as shown. The offset formation allows for more devices per wafer, with the possibility of slightly lower device uniformity for devices closer to the indicated inner ring of the wafer's 5 mm edge exclusion.

Embodiments of the present disclosure may include a microelectrode array that is communicatively coupled to a high-density interconnect or quick connect connector. For example, a high-density interconnect may include a connector or interposer that is configured to translate between the microelectrode array and an external cable assembly. In some embodiments, the microelectrode array may include a region of monolithic connectors including a set of pads that are monolithically fabricated alongside the rest of the array at one end of the flexible cable. The external cable assembly may be composed of a printed circuit board (PCB) or other electronic device, such that the high-density pads in the microelectrode array having a high-channel count can be robustly connected to another external interface for data processing, power transfer, or other purposes.

The high-density connector may be manufactured by a process that includes the step of pressing and sintering paste to form a planar metal-ceramic feedthrough, which may then be bonded to the array using eutectic or thermocompressive bonding at the feedthrough connection points, and at a portion of the perimeter. This process may form an electrical seal for the connector. Contacts of the feedthrough may be disposed along the length of a sealed elongate body such that a large number of contacts are disposed along a distance according to the pitch and number of the contacts. The contacts can be deposited on a template for an elongate body by thin film deposition techniques such as plating or sputtering, and patterning via dissolution of a sacrificial layer, deposition and etching with a masking layer, and/or direct ablation. In some embodiments a flat template may be used. The flat template may be rolled and fused to form a continuously sealed elongate body, in the form of a tube that can mate with another elongate connector body with similar configuration. A sealing ring can be patterned and located at one end of the elongate body for physical sealing by radial force.

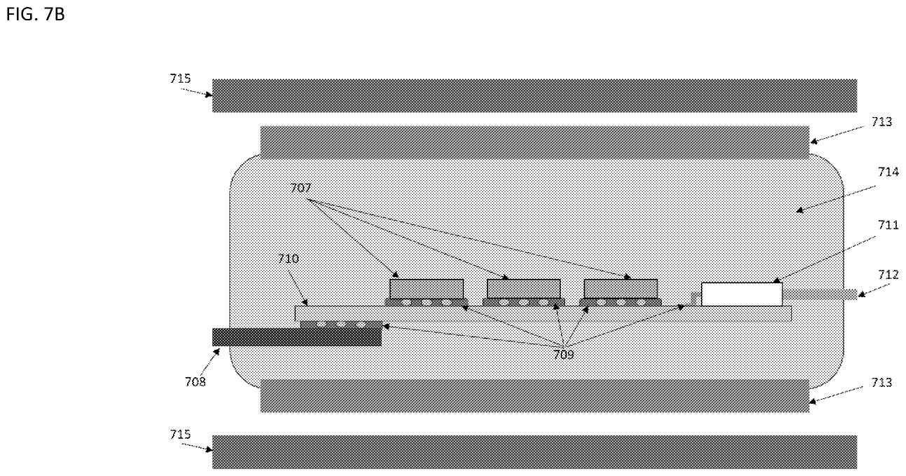

FIG. 7A provides a schematic view of the thin-film based feedthrough on the array 701 and tube side 702 in accordance with an embodiment. Contacts 703 may be disposed along wing-like features similar to the microelectrode array wings, with edges A and B on either side of the connector electrode array. Analogous to the microelectrode array fabrication process described in connection with FIG. 4A, the contacts and wing-like features may be monolithically fabricated alongside the rest of the array. As illustrated, the electrodes may include pad metal to provide for better contact to the tube side of the feedthrough. Sealing rings 704 may also be fabricated in the same pad metal layer. To create the tubular feedthrough structure, edges A and B may be wrapped into a cylindrical shape to mate and may be fused at their edges using thermal or adhesive means. The mating portion of the sealing rings can be filled with conductive material using plating, conductive paste, or other means to form a continuous ring. The same process can be used to form another thin-film that can be wrapped around the outside of a tube and similarly sealed to form the tube/thin-film feedthrough component 702 with electrodes 706 and seal rings 707. Sliding component 705 over component 702 to mate the sealing rings provides a metal-to-metal seal around each ring, and the feedthrough can be completely sealed by eutectic mixtures, thermocompression, or simple pressure sealing across the metal rings. The mating of the electrodes can be achieved in the same step by any of these methods.

In some embodiments, the connector may be manufactured to be capable of being flexed within a tube. For example, in some embodiments the conductive regions of the feedthrough may be formed using an anisotropic conductive adhesive film, with partially isolated conductive components (e.g., nanomaterial conductive components) for reduced lateral conductivity at fine pitch, and with flexibility of the interface maintained by the shape of the anisotropic conductive film at the connection interface. In some embodiments, fast contact can be made across contact interfaces by insertion in a connector that applies force needed to make contact across this film. In some embodiments, lateral conductivity can be limited by conduction across a thin insulating film with appropriate energy band alignment or electronic structure for selective carrier transport primarily across the dimension bridging two aligned contacts.

In some embodiments, while conventional connectors may require a direct sandwich stack of ADC, thin-film, and PCB, the disclosed connector may utilize a more traditional configuration with a single sided bond interface similar to the flex-to-board attach methods that are used on displays and some smartphone PCB interconnects. However, the disclosed connector may utilize a full ball grid array rather than a single row of contacts. For example, the disclosed connectors may include chips that are positioned to be directly opposite to the array, but with a normal PCB between them rather than the through hole structure utilized in some conventional connectors. In some embodiments, the disclosed systems may include a PCB and connector configured to interface with the flexible surface microelectrode arrays. In some embodiments, the connector may be sealed. For example, the connector may be configured to allow a direct connection between the interior and exterior of the body while sealing the connecting area to prevent unwanted portions of the interior being transported to the exterior and/or unwanted portions of the exterior being transported to the interior. In one embodiment, the connection between the interior and exterior is a fluidic connection. In another embodiment, the connection between the interior and exterior is an electrical connection.

In some embodiments, the feedthrough may be comprised of two components which slide into each other to form a seal. When the two pieces mate, the forces each impart on the other hold the feedthrough in place through a radial force being generated and/or an axial clamping force. In some embodiments, the two-component feedthrough contact area may utilize an attachment method in addition to radial force, such as laser and/or thermal bonding, multilayer reactive bonding, chemical bonding, or other similar methods. In other embodiments, the feedthrough may be comprised of a single piece that fits around the connection and seals around the connection and/or the body as the feedthrough is inserted into the body. The mating process may be similar to that described in FIG. 7A with relation to elements 702 and 705.

FIG. 7B depicts a cross-section of an encapsulated connector region within a catheter in accordance with an embodiment. In some embodiments, the disclosed neural interfaces may utilize a catheter-based near-hermetic connector with an indwelling assembly. For example, the connection scheme may utilize a connector with near-hermetic conditions where the flexible array cable is connected to stiffened flexible cable portions. The connector region may or may not include integrated circuit chips 707 that are attached to the connector region of a microelectrode array 708 via a bump, wire, or ball bonded connections 709 and interposer printed circuit board 710. The interposer printed circuit board 710 may be fabricated using standard PCB techniques or flex-PCB techniques, and the microelectrode array 708 may include the connector region with bond pads fabricated in an analogous manner to that described above. A standard or custom connector 711 and cable 712 can be used for output signals. All of these connections may be fully encapsulated within a stabilizing sheath 713 which is filled with a compliant insulator and dielectric 714 such as a silicone or fluoroelastomer to enhance the compliance and stability of the connected portions. The stabilizing sheath can be composed of a similar material to the deployment catheter 715 and can have an inner diameter designed to circumscribe the total dimensions of the connected stack, and an outer diameter sized to fit within the deployment catheter 715. The compliant insulator can extend along the distance of the compliant sheath on either end of the connectorized region for a distance necessary to provide mechanical stability as well as protection against corrosion, spanning for example, from millimeters to centimeters in scale.

The proximal side of the microelectrode array may be configured to connect to electronic leads that connect to electronics located outside of the brain. In some embodiments, the distal side of the microelectrode array may be configured to engage with the brain tissue.

In some embodiments, electrodes from the microelectrode array may be connected with conductive traces within a flexible cable. The flexible cable may then extend into a biocompatible housing or connector. The biocompatible housing or connector may include an analog-to-digital converter (ADC), printed circuit board (PCB), a multiplexer, interposer printed circuit boards with pads to bond to the electronic components and the flexible cable, and the like. In some embodiments, the biocompatible housing or connector may relay information to an external device, such as one configured to record from or send signals to the microelectrode array.

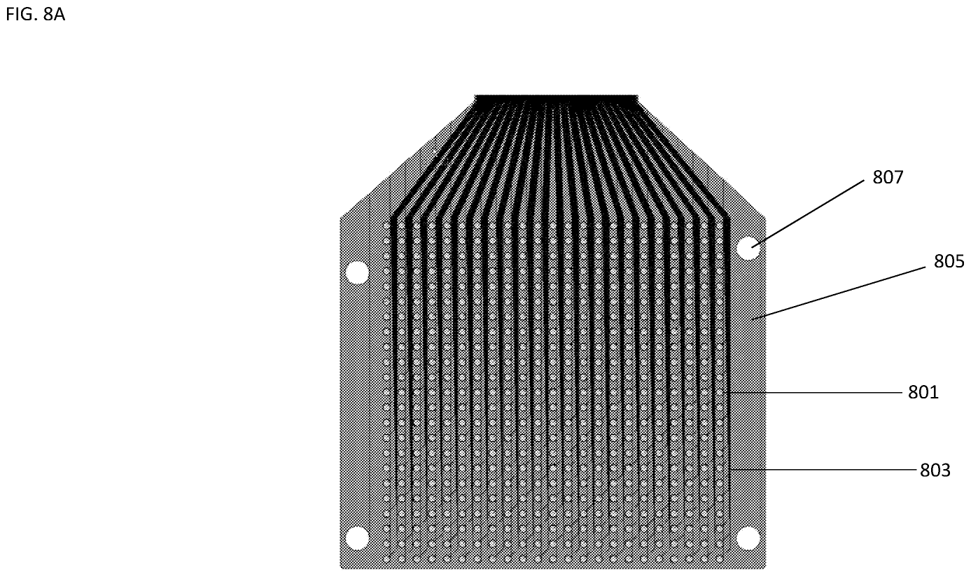

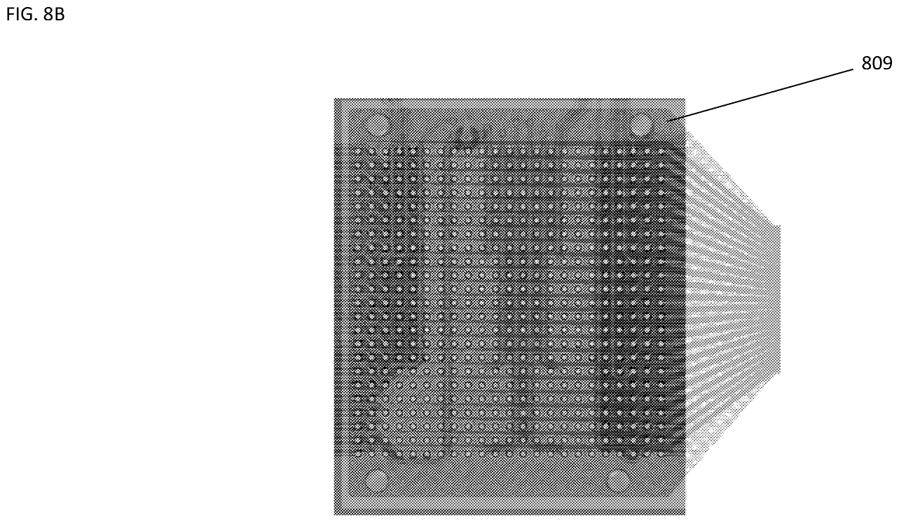

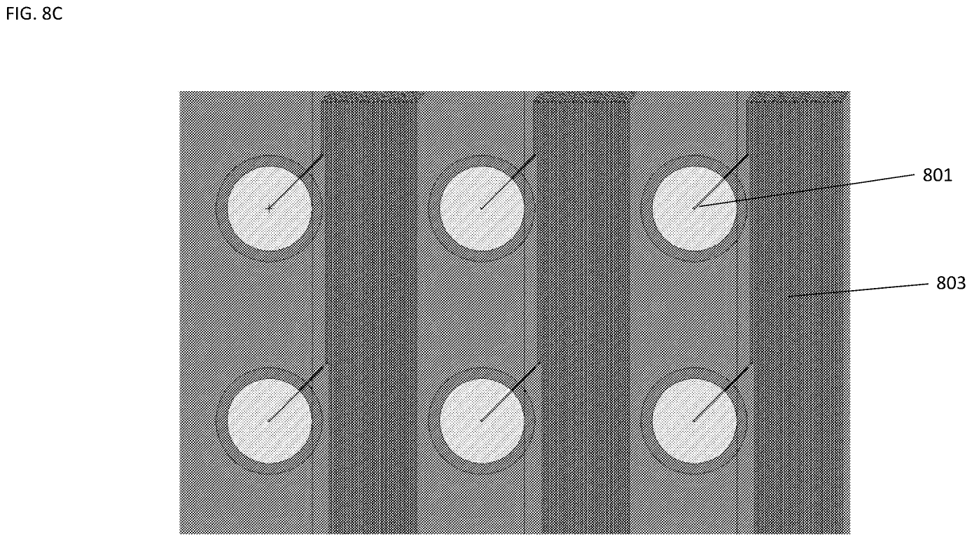

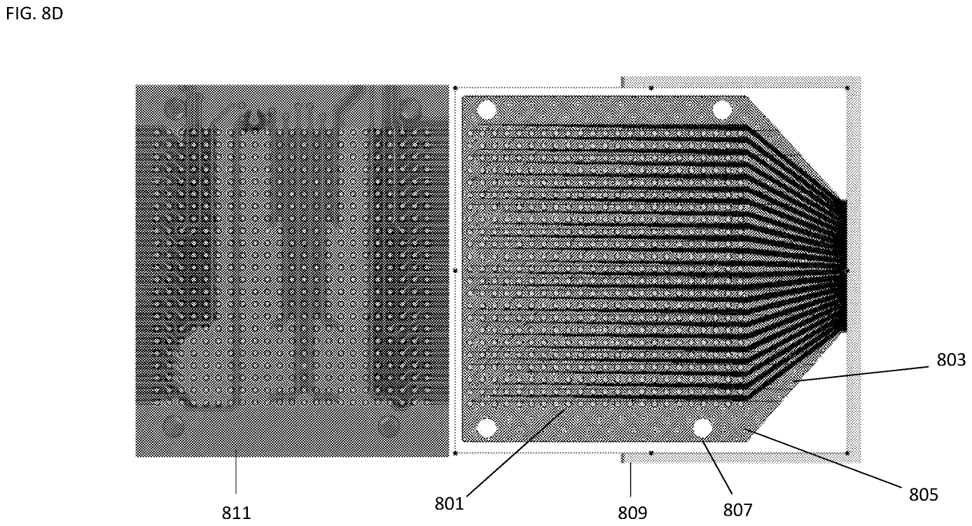

FIGS. 8A-8D depict examples of a printed circuit board (PCB) and connector for a flexible surface microelectrode array in accordance with various embodiments. For example, a printed circuit board and connector is illustrated in FIG. 8A, from the proximal end of the microelectrode array. As depicted, the microelectrode array may include a plurality of electrodes 801 connected to traces 803. The microelectrode array may be composed of thin-film 805 and include alignment holes or eyelets 807 for engaging with a connector PCB 809, illustrated in FIG. 8B.

FIG. 8C provides a close up of an electrode pad within the printed circuit board (PCB) in accordance with an embodiment. As illustrated electrodes 801 may be connected to traces 803. As illustrated electrodes may have a pad size of between about 20-250 microns in diameter.

FIG. 8D provides a schematic for a PCB-connector assembly for the flexible surface array in accordance with an embodiment. As shown, the PCB 811 may include alignment holes configured to position the PCB with the flexible surface array including the electrodes 801 having traces 803 covered in thin-film 805 with eyelets 807 for engaging with the stent-mesh 809.

In some embodiments, a connector for neural interfaces includes a biocompatible housing configured to receive conductive traces embedded in a flexible cable extending from a microelectrode array, a hermetically sealed feedthrough, an analog-to-digital converter (ADC), a printed circuit board (PCB), and a multiplexer. In some embodiments, the biocompatible housing may include a sealed metal can and/or a polymer or metal cannula with embedded and sealed electronic components. As described herein, the hermetically sealed feedthrough may include an array of metal feedthroughs co-sintered with a ceramic insulator. For example, in some embodiments, the hermetically sealed feedthrough may be configured to include electronic components that are embedded a distance of at least 5 millimeters from the ends of the cannula. Further, the hermetically sealed feedthrough may be encapsulated by an elastomeric barrier material. Examples of the elastomeric barrier material may include (without limitation) silicones and/or fluroelastomers. In some embodiments, the ADC, PCB and multiplexer components of the connector may be fabricated and bonded using the flex-hybrid techniques and substrates described herein. The components of the connector may be constructed to provided limited flexibility while they are embedded within the cannula and barrier material. In some embodiments, the PCB may be fabricated to be partially or fully flexible. In some embodiments, the ADC and/or multiplexer may be thinned in order to be partially flexible. In some embodiments, the ADC and/or the multiplexer may be packaged using semiconductor-on-polymer technology to have a minimum radius of curvature less than the radius of curvature of the anticipated bending within the connector during packaging under normal operation. In some embodiments, the connector for the neural interface may include a hermetically sealed feedthrough having an electrode array with electrodes disposed along the lateral wing appendages of the electrode array and a central region coaxial with a cable. Further, the wing appendages may be sealed into a tubular configuration with electrodes disposed along the inner surface of the tube. Sealing rings may be applied to each end of the electrode array and sealed by metal-to-metal contact. For example, metal to metal contact may be provided by expanding a rod inside the two mating features. The sealing mechanism may include a thermally shrinkable tube that mates the two metal surfaces and all the electrodes when shrunk together with a central mandrel, with the possible addition or separate usage of eutectic or thermal compression bonding between all the metal interfaces. The two metal surfaces may be aligned using optical, or mechanical features on the electrode arrays.

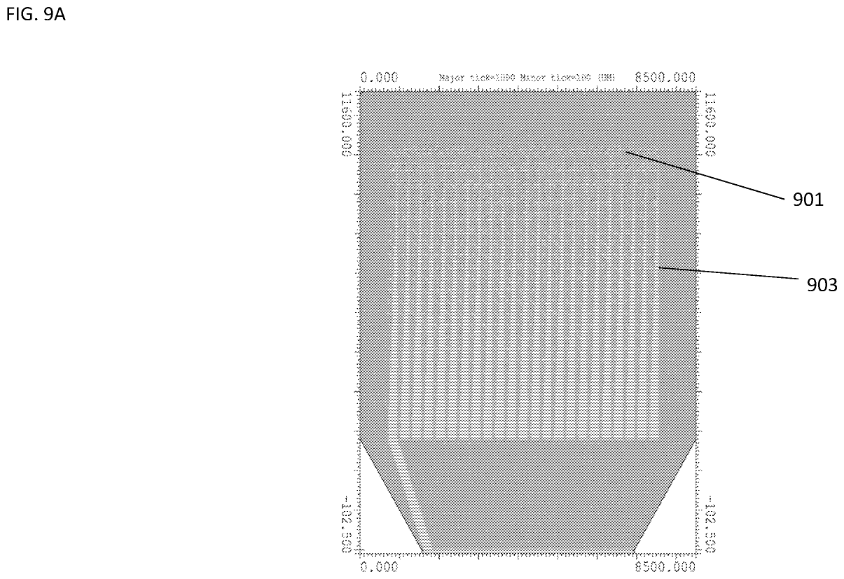

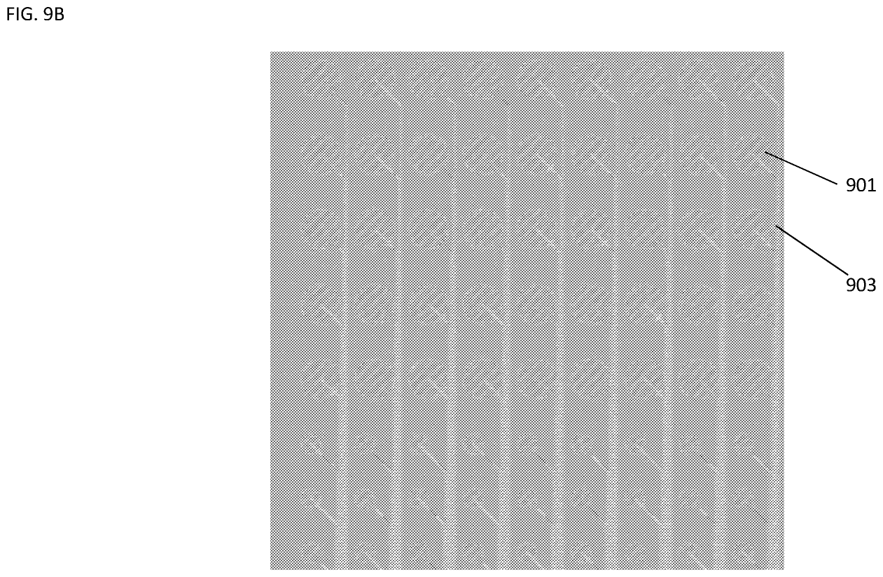

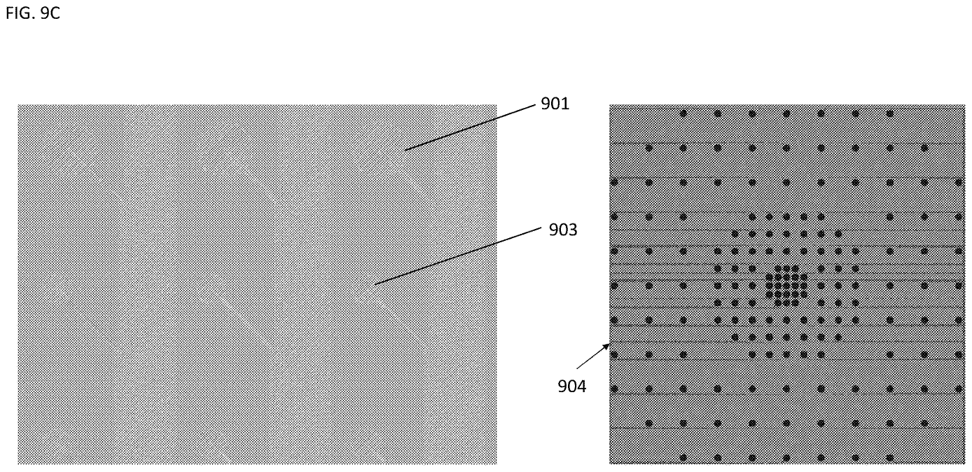

Referring now to FIGS. 9A-9C, various views of a surface array layout are depicted in accordance with an embodiment. FIG. 9A provides a first view of a surface array layout for a microelectrode array in accordance with an embodiment. FIG. 9B provides a close-up view of the surface array layout in accordance with an embodiment. FIG. 9C provides a second close-up view of the surface array layout in accordance with an embodiment. As shown, electrode pads 901 may be connected via electrical traces 903. Electrode pads can have various or uniform sizes and various or uniform pitches; the exemplified varying size and uniform pitch can, for example, identify size-performance variation. FIG. 9C also provides an example of an additional pad layout 904, having foveated designs. The additional pad layout 904 displays varying resolution within a given electrode cluster, which can be duplicated in a subarray along the body of a given overall microelectrode array.

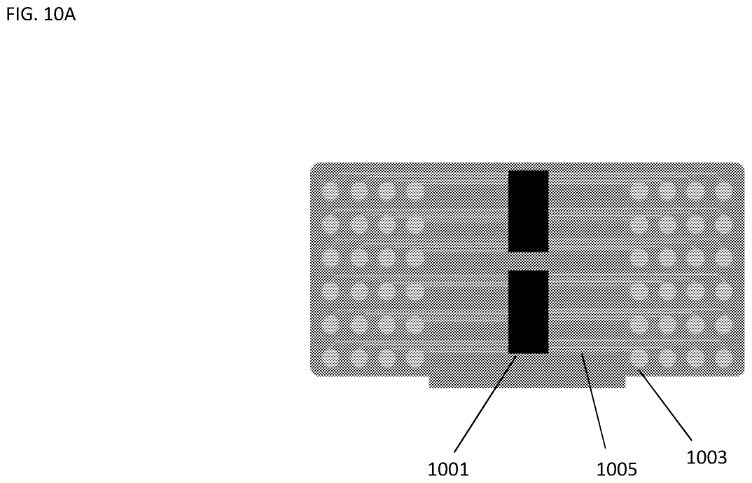

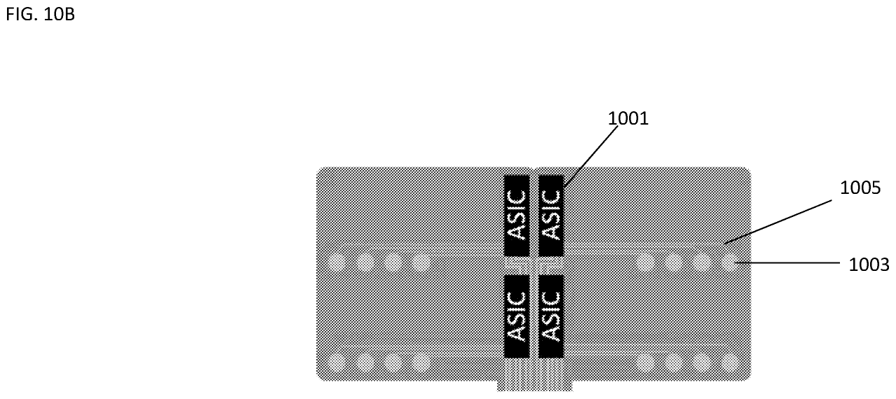

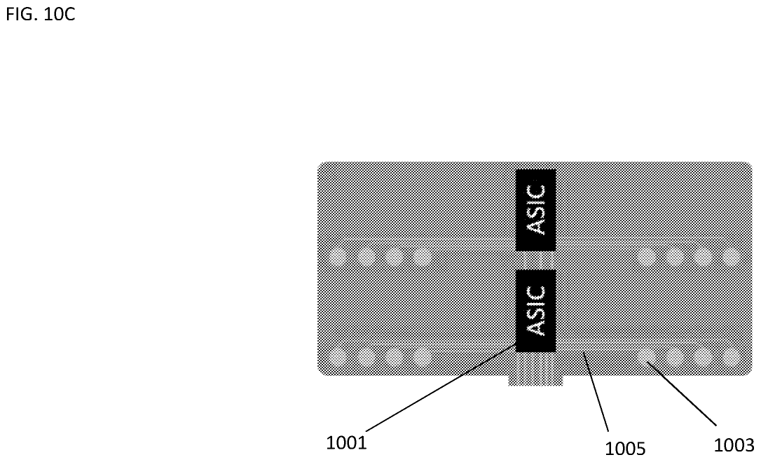

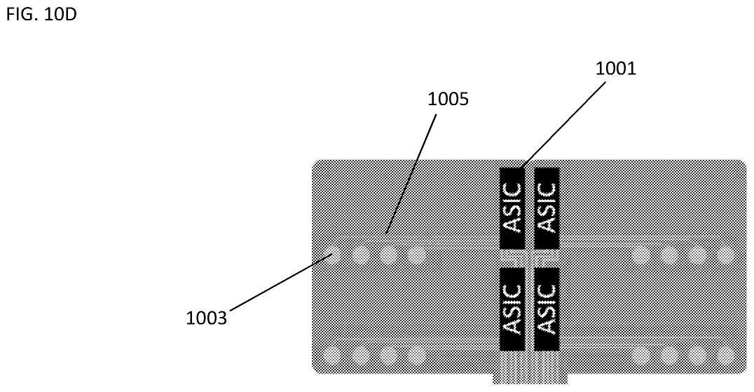

Referring now to FIGS. 10A-10D, various surface array schematic layouts with multiplexing elements are depicted in accordance with an embodiment. As shown, an application specific integrated circuit (ASIC) 1001 may be connected via traces 1005 to microelectrode arrays 1003. For example, FIG. 10A illustrates two centrally located ASIC components 1001 with corresponding microelectrode arrays 1003 connected thereto. FIG. 10B illustrates a second configuration for a surface array with multiplexing elements in accordance with an embodiment. FIG. 10C illustrates a third configuration for a surface array with multiplexing elements in accordance with an embodiment. FIG. 10D illustrates a fourth configuration for a surface array with multiplexing elements in accordance with an embodiment. The various surface array schematics provide for varied size and spacing of electrodes on the array 1003. The ASIC 1001 itself may have limited surface area for connecting with the microelectrode arrays 1003.

In some embodiments, the neural interfaces described herein may include actively multiplexed, catheter-deployed electrode arrays. For example, the active microelectrode array may be composed of thin-film microelectrodes. The active microelectrode array may also be attached to a spring-like mesh analogous to that described above.

The thin-film electrodes and thin-film active electronics body may include two lateral wing-like appendages located distal to a thin-film flex-cable that is terminated at the proximal end in a thin-film connector region. The thin-film flex-cable may be fabricated from a single piece, similar to the fabrication process described above with respect to the thin-film microelectrode array.

In some embodiments, the thin-film active electronics body may include microelectrodes and active optical and/or electronic devices located at least partly on the wing appendages and/or at least partly on the central region of the electrode body coaxial with the flex-cable.

Analogous to the wings described above, the thin-film active electronics body may include two lateral wings configured to be curled into a loose spiral state when inserted into a delivery catheter or other confined space. In some embodiments, the thin-film flex-cable and coaxial region of the microelectrode array can be flat or slightly curved in the same direction as the lateral wings for co-insertion in a delivery catheter or other confined space. In some embodiments, the thin-film active electronics body may include an application-specific integrated circuit (ASIC) attached in the flex-cable, coaxial flat region, or wing regions of the array using flex-hybrid packaging and assembly techniques. These flex-hybrid techniques may include, but are not limited to, chip thinning techniques such as back grinding or etching with dicing and chip transfer, anisotropic conductive adhesive or film bonding, and flexible overmolding co-designed to provide limiting radius of curvature below the anticipated radius of curvature experienced by the bond region in a given deployment mechanism.

In some embodiments, the thin-film active electronics body may be composed of one or more or low-temperature polycrystalline silicon (LTPS), low-temperature polycrystalline oxide (LTPO), inorganic semiconductor thin-film transistors (TFT), and thin-film barriers.

The active electronics body can include flexible active electronic circuits (for example multiplexers, amplifiers, analog to digital converters, filters, memory components, and sensors) and flexible active optoelectronics (for example optical sensors and optical emitters). These components can be driven and controlled independently, or function interactively, and can be used for recording and stimulation from tissue with or without feedback.

In some embodiments, a microelectrode array with embedded active optical and/or electronic circuits can be fabricated using microfabrication steps including, but not limited to, spin-coating and curing or lamination of a polymer insulator base layer, deposition of a first barrier layer, deposition and patterning of at least one type of thin-film organic or inorganic semiconductor (active layer), deposition and patterning of a gate dielectric layer, deposition and patterning of source/drain contacts, deposition and patterning of a second barrier layer and interlayer dielectric fully encapsulating the active layer, deposition and patterning of one or more layers of trace/electrode/pad metal, spin coating and curing or lamination of a polymer insulator top layer, patterning and/or etching of the top polymer layer to expose at least some of the pad and trace metal layer, and optional plating or deposition and patterning of a top pad or electrode material layer. The pad and trace metals of the microelectrode array with embedded active circuits may include titanium, platinum, and gold for adhesion or conduction properties. The electrode material for the microelectrode array may include organic conductive materials such as PEDOT:PSS or inorganic conductive materials and metals such as platinum, iridium and its compounds, ruthenium and its compounds, and titanium nitride. The polymer insulator of the microelectrode array may include thermoplastics, thermoset polymers, liquid crystal polymers, semi-crystalline polymers, and elastomers including polyimide, polyetherimide (PEI), silicones, fluoroelastomers, polyurethanes, and Parylenes. The active semiconductor components of the microelectrode arrays may include low temperature polysilicon or low temperature oxide semiconductors and organic or inorganic optical emitters, and can be designed specifically in dimensions or material properties to ensure functionality at the radius of curvature required for the described deployment method, whether in the central or wing region of the array. The gate dielectric of the microelectrode arrays may be any combination of aluminum oxide, zirconium oxide, hafnium oxide, silicon nitride, silicon oxynitride, silicon carbide, and additional ceramic materials, and can be deposited by sputtering, atomic layer deposition, or chemical vapor deposition. The insulating barriers encapsulating the active layer components of the microelectrode array may include any combination of aluminum oxide, hafnium oxide, zirconium oxide, silicon nitride, and silicon carbide, and can be deposited by sputtering, atomic layer deposition, or chemical vapor deposition.

Methods for manufacturing and fabricating the thin-film neural interfaces described herein fabricating thin-film neural interfaces on glass panels using thin-film photovoltaics or display processes or a roll-to-roll process. For example, a method for fabricating thin-film neural interfaces having a scale in the range of centimeters or meters may include integrated polymer flex cables positioned on large area glass panels. The fabrication process may utilize belt-driven panel-scale process equipment that substitutes wafer-scale processing techniques such as polymer solution blade coating equipment (replacing polyimide, photoresist spin coating), panel-scale stepper photolithography exposure tools, belt-driven or large format physical vapor deposition tools at atmospheric or high vacuum pressure for metal and ceramic coatings (replacing wafer scale electron beam or thermal evaporation equipment for metal or ceramic coating), and belt-driven or large format chemical and/or plasma etching and development tools (replacing small bath chemical etching/development or wafer-scale vacuum plasma etching tools). The overall fabrication process can be similar to both the passive and active descriptions above, but with appropriate tooling typically used in display panel fabrication adapted to our application.

In some embodiments, a method of fabricating thin-film neural interfaces having centimeter to meter scale integrated polymer flex cables on flexible polymer foils may utilize belt-driven roll-to-roll process equipment. Example tools and equipment needed for fabricating such thin-film neural interfaces may include, without limitation, slot-die or gravure coating (e.g., polymer precursors/films, including resists), roll or belt fed furnaces (e.g., polymer cure, other anneals), roll or belt fed sputtering, evaporation tools (e.g., typically low vacuum), and/or roll or belt fed photolithography (e.g., direct write laser only or panel scale steppers).

Embodiments built in accordance with the present disclosure include flexible arrays with or without active electrodes. The flexible arrays may be designed and configured for self or assisted deployment such that the arrays are compatible with the anticipated radius of curvature, conformality and mechanical requirements to roll and unroll from a deployment device. In some embodiments, the rolling and unrolling process may include a stent-mesh assembly. In some embodiments, the flexible array may unroll to conform to structures within the brain, and may be deployed through an angled cranial incision.

Embodiments built in accordance with the present disclosure may also include flexible arrays having embedded or attached active electronics. The active electronic components may be encapsulated and designed for flexibility such that they can operate within the neural environment. In some embodiments, the embedding and co-encapsulation of the active electronic components may be achieved by thin-film semiconductors that are embedded in the thin-film during fabrication. In some embodiments, the attachment and encapsulation of the active electronic components is achieved by flex-hybrid thinning, attaching to the array body, and encapsulation methods.

Embodiments built in accordance with the present disclosure include a neural interface device comprising a thin-film microelectrode array configured to at least one of record from or stimulate a target area. In some embodiments the thin-film microelectrode array may be configured to be inserted through an angled cranial incision, and then to conform to brain structures upon contact with the brain. The thin-film microelectrode array may include a connector, a thin-film flexible cable in electrical communication with the connector, and two lateral wings distal to the thin-film flexible cable. Optionally, the two lateral wings may be flexible. Optionally, the connector may include a hermetically sealed feedthrough. Optionally, the thin-film microelectrode array may include active electronics, where the active electronics may have a rigid or flexible application-specific integrated circuit bonded to the array body and encapsulated. Optionally, the thin-film microelectrode array with active electronics may include an integrated circuit fabricated monolithically along the thin-film microelectrode array using thin-film semiconductors.

Additionally, embodiments built in accordance with the present disclosure may include connectors that are designed to enable passive connections or attachment or embedding of active electronics with the flexible array.

### Data Processing Systems for Implementing Embodiments Herein

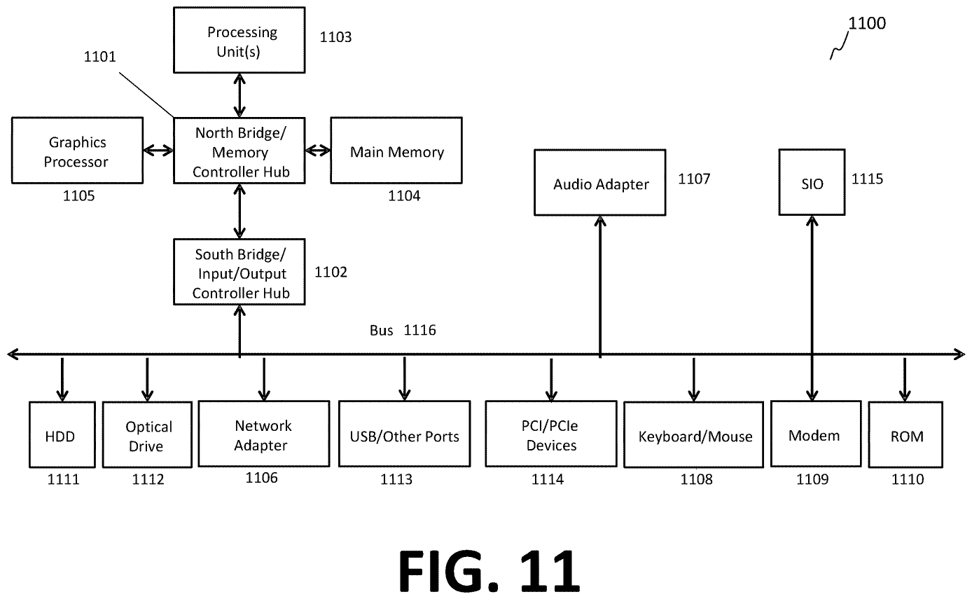

FIG. 11 illustrates a block diagram of an exemplary data processing system 1100 in which embodiments are implemented. The data processing system 1100 is an example of a computer, such as a server or client, in which computer usable code or instructions implementing the process for illustrative embodiments of the present invention are located. In some embodiments, the data processing system 1100 may be a server computing device. For example, data processing system 1100 can be implemented in a server or another similar computing device operably connected to a neural device 100 with respect to FIG. 1 or a system as described with respect to FIGS. 6A-6C and/or 7A-7B. The data processing system 800 can be configured to, for example, transmit and receive information or signals related to a subject between the devices described herein and an external device.

In the depicted example, data processing system 1100 can employ a hub architecture including a north bridge and memory controller hub (NB/MCH) 1101 and south bridge and input/output (I/O) controller hub (SB/ICH) 1102. Processing unit 1103, main memory 1104, and graphics processor 1105 can be connected to the NB/MCH 1101. Graphics processor 1105 can be connected to the NB/MCH 1101 through, for example, an accelerated graphics port (AGP).

In the depicted example, a network adapter 1106 connects to the SB/ICH 1102. An audio adapter 1107, keyboard and mouse adapter 1108, modem 1109, read only memory (ROM) 1110, hard disk drive (HDD) 1111, optical drive (e.g., CD or DVD) 1112, universal serial bus (USB) ports and other communication ports 1113, and PCI/PCIe devices 1114 may connect to the SB/ICH 1102 through bus system 1116. PCI/PCIe devices 1114 may include Ethernet adapters, add-in cards, and PC cards for notebook computers. ROM 1110 may be, for example, a flash basic input/output system (BIOS). The HDD 1111 and optical drive 1112 can use an integrated drive electronics (IDE) or serial advanced technology attachment (SATA) interface. A super I/O (SIO) device 1115 can be connected to the SB/ICH 1102.

An operating system can run on the processing unit 1103. The operating system can coordinate and provide control of various components within the data processing system 1100. As a client, the operating system can be a commercially available operating system. An object-oriented programming system, such as the Java^{TM} programming system, may run in conjunction with the operating system and provide calls to the operating system from the object-oriented programs or applications executing on the data processing system 1100. As a server, the data processing system 1100 can be an IBM® eServer™ System® running the Advanced Interactive Executive operating system or the Linux operating system. The data processing system 1100 can be a symmetric multiprocessor (SMP) system that can include a plurality of processors in the processing unit 1103. Alternatively, a single processor system may be employed.

Instructions for the operating system, the object-oriented programming system, and applications or programs are located on storage devices, such as the HDD 1111, and are loaded into the main memory 1104 for execution by the processing unit 1103. The processes for embodiments described herein can be performed by the processing unit 1103 using computer usable program code, which can be located in a memory such as, for example, main memory 1104, ROM 1110, or in one or more peripheral devices.

A bus system 1116 can be comprised of one or more busses. The bus system 1116 can be implemented using any type of communication fabric or architecture that can provide for a transfer of data between different components or devices attached to the fabric or architecture. A communication unit such as the modem 1109 or the network adapter 1106 can include one or more devices that can be used to transmit and receive data.

Those of ordinary skill in the art will appreciate that the hardware depicted in FIG. 11 may vary depending on the implementation. Other internal hardware or peripheral devices, such as flash memory, equivalent non-volatile memory, or optical disk drives may be used in addition to or in place of the hardware depicted. Moreover, the data processing system 1100 can take the form of any of a number of different data processing systems, including but not limited to, client computing devices, server computing devices, tablet computers, laptop computers, telephone or other communication devices, personal digital assistants, and the like. Essentially, data processing system 1100 can be any known or later developed data processing system without architectural limitation.

In the above detailed description, reference is made to the accompanying drawings, which form a part hereof. In the drawings, similar symbols typically identify similar components, unless context dictates otherwise. The illustrative embodiments described in the present disclosure are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Instead, this application is intended to cover any variations, uses, or adaptations of the present teachings and use its general principles. Further, this application is intended to cover such departures from the present disclosure as come within known or customary practice in the art to which these teachings pertain. Many modifications and variations can be made to the particular embodiments described without departing from the spirit and scope of the present disclosure as will be apparent to those skilled in the art. Functionally equivalent methods and apparatuses within the scope of the disclosure, in addition to those enumerated herein, will be apparent to those skilled in the art from the foregoing descriptions. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

As used in this document, the singular forms “a,” “an,” and “the” include plural references unless the context clearly dictates otherwise. With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

As will be understood by one skilled in the art, for any and all purposes, such as in terms of providing a written description, all ranges disclosed herein are intended as encompassing each intervening value between the upper and lower limit of that range and any other stated or intervening value in that stated range. All ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells as well as the range of values greater than or equal to 1 cell and less than or equal to 3 cells. Similarly, a group having 1-5 cells refers to groups having 1, 2, 3, 4, or 5 cells, as well as the range of values greater than or equal to 1 cell and less than or equal to 5 cells, and so forth.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by hereby reserving the right to proviso out or exclude any individual substituents, structures, or groups thereof, or any members of a claimed group, less than the full measure of this disclosure can be claimed for any reason.

The term “about,” as used herein, refers to variations in a numerical quantity that can occur, for example, through measuring or handling procedures in the real world; through inadvertent error in these procedures; through differences in the manufacture, source, or purity of compositions or reagents; and the like. Typically, the term “about” as used herein means greater or lesser than the value or range of values stated by 1/10 of the stated values, e.g., ±10%. The term “about” also refers to variations that would be recognized by one skilled in the art as being equivalent so long as such variations do not encompass known values practiced by the prior art. Each value or range of values preceded by the term “about” is also intended to encompass the embodiment of the stated absolute value or range of values. Whether or not modified by the term “about,” quantitative values recited in the present disclosure include equivalents to the recited values, e.g., variations in the numerical quantity of such values that can occur, but would be recognized to be equivalents by a person skilled in the art. Where the context of the disclosure indicates otherwise, or is inconsistent with such an interpretation, the above-stated interpretation may be modified as would be readily apparent to a person skilled in the art. For example, in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). Further, the transitional term “comprising,” which is synonymous with “including,” “containing,” or “characterized by,” is inclusive or open-ended and does not exclude additional, unrecited elements or method steps. While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

The terms “patient” and “subject” are interchangeable and refer to any living organism which contains neural tissue. As such, the terms “patient” and “subject” may include, but are not limited to, any non-human mammal, primate or human. A subject can be a mammal such as a primate, for example, a human. The term “subject” includes domesticated animals (e.g., cats, dogs, etc.); livestock (e.g., cattle, horses, swine, sheep, goats, etc.), and laboratory animals (e.g., mice, rabbits, rats, gerbils, guinea pigs, possums, etc.). A patient or subject may be an adult, child or infant.

The term “tissue” refers to any aggregation of similarly specialized cells which are united in the performance of a particular function.

The term “disorder” is used in this disclosure to mean, and is used interchangeably with, the terms “disease,” “condition,” or “illness,” unless otherwise indicated.

The term “real-time” is used to refer to calculations or operations performed on-the-fly as events occur or input is received by the operable system. However, the use of the term “real-time” is not intended to preclude operations that cause some latency between input and response, so long as the latency is an unintended consequence induced by the performance characteristics of the machine.

Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

Throughout this disclosure, various patents, patent applications and publications are referenced. The disclosures of these patents, patent applications and publications are incorporated into this disclosure by reference in their entireties in order to more fully describe the state of the art as known to those skilled therein as of the date of this disclosure. This disclosure will govern in the instance that there is any inconsistency between the patents, patent applications and publications cited and this disclosure.

What is claimed is:

1. A neural interface for implantation against a cortical surface of a brain of a subject comprising:
a flexible substrate that resides on a target region of the cortical surface of the brain; and
a thin-film microelectrode array configured to record from the target region, the thin-film microelectrode array comprising:
a first insulating layer disposed on the flexible substrate;
a trace metal layer disposed on the first insulating layer;
a second insulating layer disposed on the trace metal layer; and
an electrode layer disposed on the second insulating layer that contacts the trace metal layer and records from the target region of the cortical surface.

2. The neural interface of claim 1, wherein at least one of the first insulating layer and the second insulating layer comprise polyimide.

3. The neural interface of claim 1, wherein the trace metal layer comprises titanium, platinum, or gold.

4. The neural interface of claim 1, wherein the electrode layer comprises titanium, platinum, gold, iridium, or ruthenium.

5. The neural interface of claim 1, further comprising an insulating adhesion layer disposed between the first insulating layer and the trace metal layer, wherein the insulating adhesion layer comprises silicon oxide, silicon nitride, silicon oxynitride, silicon carbide, silicon oxycarbide, or transition metal oxide.

6. The neural interface of claim 1, further comprising a pad metal layer disposed between the second insulating layer and the electrode layer.

7. The neural interface of claim 1, wherein the thin-film microelectrode array is configured to be deployed on the cortical surface to treat epilepsy, paralysis associated with stroke or spinal cord injury, visual impairment, Parkinson's disease, or cognitive impairment.

8. The neural interface of claim 1, wherein the thin-film microelectrode array is configured to be deployed on the cortical surface to record neural activity for diagnosis or treatment of epilepsy, seizure disorders, or cognitive impairment.

9. A method for fabricating a microelectrode array, the method comprising:
depositing a first insulating layer onto a substrate;
depositing a trace metal layer onto the first insulating layer;
depositing a second insulating layer onto the trace metal layer;
patterning the second insulating layer to expose a region of the trace metal layer; and
depositing an electrode layer onto the second insulating layer, wherein the electrode layer contacts the exposed region of the trace metal layer.

10. The method of claim 9, wherein depositing each of the first insulating layer and the second insulating layer comprises spin-coating polyimide and curing.

11. The method of claim 9, wherein depositing the trace metal layer comprises depositing titanium, platinum, or gold using photolithography and metal deposition.

12. The method of claim 9, wherein depositing the electrode layer comprises depositing titanium, platinum, gold, iridium, or ruthenium.

13. The method of claim 9, further comprising generating an insulating adhesion layer between the first insulating layer and the trace metal layer using chemical processing or vapor deposition, wherein the insulating adhesion layer comprises silicon oxide, silicon nitride, silicon oxynitride, silicon carbide, silicon oxycarbide, or transition metal oxide.

14. The method of claim 9, wherein depositing the electrode layer comprises photolithography, deposition, and liftoff to form a plurality of electrodes.

15. The method of claim 9, further comprising:
deploying the microelectrode array on a cortical surface of a brain of a subject; and
using the microelectrode array to treat epilepsy, paralysis associated with stroke or spinal cord injury, visual impairment, Parkinson's disease, or cognitive impairment.

16. The method of claim 9, further comprising:
deploying the microelectrode array on a cortical surface of a brain of a subject; and
using the microelectrode array to record neural activity for diagnosis or treatment of epilepsy, seizure disorders, or cognitive impairment.
